(19) United States
(12) Patent Application Publication
(10) Pub. No.: US 2025/0052746 A1
(43) Pub. Date: Feb. 13, 2025

(54) DIAGNOSTIC ASSAY USING MAGNETIC PARTICLES

(71) Applicant: OSLER DIAGNOSTICS LIMITED, Oxford (GB)

(72) Inventors: Konstantinos PSARROS, Oxford (GB); Peter READER, Oxford (GB); Fatiha TAYLOR, Oxford (GB); Laure CARRIQUE, Oxford (GB); Vincent LINDEN, Oxford (GB); Barbara SIMOES, Oxford (GB); Theo VOGIAT-ZOGLOU, Oxford (GB); Maryam Noor ALI, Oxford (GB)

(21) Appl. No.: 18/722,897

(22) PCT Filed: Dec. 22, 2022
(86) PCT No.: PCT/EP2022/087657

§ 371 Date: Jun. 21, 2024

(30) Foreign Application Priority Data
Dec. 23, 2021 (GR) 20210100910
Mar. 14, 2022 (GB) 2203535.6

(51) Int. Cl. G01N 33/543; C12Q 1/28; C12Q 1/42; G01N 33/68

(52) U.S. Cl. CPC G01N 33/54326; C12Q 1/28; C12Q 1/42; G01N 33/5438; G01N 33/68; G01N 2333/58; G01N 2333/908; G01N 2333/916; G01N 2470/04

(57) ABSTRACT

The present invention relates to an apparatus and method for determination of analytes in biological samples by immunoassays incorporating magnetic capture of beads on a sensor, capable of being used in the point-of-care diagnostic field.

The present invention relates to an apparatus and method for determination of analytes in biological samples by immunoassays incorporating magnetic capture of beads on a sensor, capable of being used in the point-of-care diagnostic field.

### BACKGROUND

Diagnostic tests, such as immunoassays, are often used for the detection of a specific analyte within a sample. For example, pairs of antibodies that can bind to an analyte to form a sandwich that is detectable by means of an enzyme or label on one or more of the antibodies are well known and available for a wide range of different analytes of interest. Antibodies to a particular biomarker, such as testosterone or cortisol, may be used to test levels of these substances in saliva, blood or urine samples. The presence of the analyte is then determined using, for example, electrochemical measurements or optical measurements, such as fluorescence. Many electrochemical measurement techniques are known to the skilled person such electrochemical impedance spectroscopy, differential pulse voltammetry, square wave voltammetry, cyclic voltammetry, chronoamperometry, open circuit potential measurement and chronopotentiometry.

Point-of-care detection brings a diagnostic test conveniently and immediately to a subject, allowing better and faster clinical decisions to be made. However, integration of diagnostic tests into a point-of-care device or system is challenging. Preparation of a sample for an immunoassay may require mixing of multiple solutions and reagents, with precise control of volumes and mixing times. Further, the device is ideally automated to obviate the need for a medical professional to be present.

Existing liquid handling devices typically flow multiple liquids (such as sample liquids, reagents or wash buffers) across measurement chambers, reaction zones or other detection means in the same flow direction (i.e. different liquids are flowed through the same conduits and parts of the device sequentially). This can cause issues with contamination since some of the liquid or reagent involved in the previous step may still be present in the conduit, measurement chambers, reaction zones or other detection means when the next liquid or reagent is added. This contamination can reduce the accuracy of the diagnostic assay.

Existing liquid handling devices which flow multiple liquids across measurement chambers, reaction zones or other detection means in the same flow direction are not capable of providing rapid, precise and controllable quenching of reactions and/or biological reactions in the measurement chambers, reaction zones or other detection means. This is because sequential linear flow of multiple reagents in the same direction does not remove the previous liquid or analyte from the measurement chambers, reaction zones or other detection means sufficiently quickly.

Thus, there is a need to provide improved immunoassay techniques that allow more accurate detection of analytes of interest and improved signal-to-noise ratios.

### SUMMARY OF THE INVENTION

This summary introduces concepts that are described in more detail in the detailed description. It should not be used to identify essential features of the claimed subject matter, nor to limit the scope of the claimed subject matter.

The present invention provides a method for measuring an analyte of interest in a biological sample comprising:
combining said biological sample with a composition comprising:
magnetically susceptible beads conjugated to a first antibody or antigen binding portion thereof capable of binding said analyte of interest; and
a second antibody or antigen binding portion thereof capable of binding said analyte of interest conjugated to an enzyme;
retaining the magnetically susceptible beads on an electrode using a magnetic field;
contacting the magnetically susceptible beads with a substrate for the enzyme, wherein the enzyme substrate is converted by the enzyme into an electroactive molecule; and
obtaining an electrochemical measurement using said electrode.

The present invention also provides a method for measuring one or more analytes of interest in a biological sample comprising:
combining said biological sample with a composition comprising:
magnetically susceptible beads conjugated to one or more antibodies or antigen binding portions thereof, wherein each antibody or antigen binding portion thereof is capable of binding one of said analytes of interest; and
one or more second antibodies or antigen binding portions thereof conjugated to an enzyme, wherein each second antibody or antigen binding portion thereof is capable of binding one of said analytes of interest;
retaining the magnetically susceptible beads on an electrode using a magnetic field;
contacting the magnetically susceptible beads with one or more substrates for the enzymes, wherein the enzyme substrates are converted by the enzymes into electroactive molecules; and
obtaining an electrochemical measurement using said electrode.

In some embodiments the method is capable of detecting at least 2, 3, 4, 5, 6, 7, 8, 9 or 10 different analytes of interest. In some embodiments, each magnetically susceptible bead is conjugated to multiple different antibodies or antigen binding portions thereof, wherein each antibody or antigen binding portion thereof is capable of binding one of said analytes of interest. In some embodiments, the magnetically susceptible beads comprise different sets of magnetically susceptible beads, wherein each set of magnetically susceptible beads is conjugated to a different antibody or antigen binding portion thereof, wherein each antibody or antigen binding portion thereof is capable of binding one of said analytes of interest.

In some embodiments, the enzyme substrate is converted by the enzyme into a soluble electroactive molecule at the electrode. In some embodiments, the enzyme substrate is converted by the enzyme into an electroactive molecule precipitated on the magnetically susceptible beads.

In some embodiments, the method further comprises:
incubating the combined biological sample and composition such that the one or more first and/or one or more second antibodies bind the analyte of interest.

In some embodiments, the method further comprises:
retaining the magnetically susceptible beads in a fixed position using a magnetic field; and
washing the magnetically susceptible beads.

In some embodiments, the fixed position is located at the electrode. In some embodiments, the fixed position is a position spatially distant from said electrode, optionally wherein the spatially distant position is a blank or non-functional electrode.

In some embodiments, the method further comprises:
retaining the magnetically susceptible beads in a first position using a magnetic field; and
washing the magnetically susceptible beads by modulating the magnetic field such that the magnetically susceptible beads are retained in a second position.

In some embodiments, the magnetic field is generated by a fixed magnet. In some embodiments, the magnetic field is generated by an electromagnet. In some embodiments, the magnetic field is modulated by physically actuating the magnet. In some embodiments, the magnetic field is modulated by controlling the current in an electromagnet.

In some embodiments, the magnetic field is located within a microfluidic device having a flow conduit and wherein the magnetic field is modulated by actuating the magnet in a direction perpendicular to the direction of flow within said flow conduit. In some embodiments, the magnetic field is located within a microfluidic device having a flow conduit and wherein the magnetic field is modulated by controlling the current in an electromagnet.

In some embodiments, the magnetically susceptible beads are moved between the first position and the second position at least 20 times, for example at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times or at least 19 times. In some embodiments, the magnetically susceptible beads are moved between the first position and the second position twice, 3 times, 4 times, 5 times, 6 times, 7 times, 8 times, 9 times, 10 times, 11 times, 12 times, 13 times, 14 times, 15 times, 16 times, 17 times, 18 times, 19 times or 20 times, preferably 12 times.

In some embodiments, the magnetic field is modulated such that the magnetically susceptible beads are moved periodically between the first position and the second position. In some embodiments, the period between the magnetically susceptible beads being in the first position and in the second position is between about 0.01 seconds to about 5 seconds, about 0.05 seconds to about 5 seconds, about 0.1 seconds to about 5 seconds, about 0.2 seconds to about 5 seconds, about 0.3 seconds to about 5 seconds, about 0.4 seconds to about 5 seconds, about 0.5 seconds to about 5 seconds, about 1 seconds to about 5 seconds, about 2 seconds to about 5 seconds, about 3 seconds to about 5 seconds, about 4 seconds to about 5 seconds, about 0.01 seconds to about 4 seconds, about 0.05 seconds to about 4 seconds, about 0.1 seconds to about 4 seconds, about 0.2 seconds to about 4 seconds, about 0.3 seconds to about 4 seconds, about 0.4 seconds to about 4 seconds, about 0.5 seconds to about 4 seconds, about 1 seconds to about 4 seconds, about 2 seconds to about 4 seconds, about 3 seconds to about 4 seconds, about 0.01 seconds to about 3 seconds, about 0.05 seconds to about 3 seconds, about 0.1 seconds to about 3 seconds, about 0.2 seconds to about 3 seconds, about 0.3 seconds to about 3 seconds, about 0.4 seconds to about 3 seconds, about 0.5 seconds to about 3 seconds, about 1 seconds to about 3 seconds, about 2 seconds to about 3 seconds, about 0.01 seconds to about 2 seconds, about 0.05 seconds to about 2 seconds, about 0.1 seconds to about 2 seconds, about 0.2 seconds to about 2 seconds, about 0.3 seconds to about 2 seconds, about 0.4 seconds to about 2 seconds, about 0.5 seconds to about 2 seconds, about 1 seconds to about 2 seconds, about 0.01 seconds to about 1 seconds, about 0.05 seconds to about 1 seconds, about 0.1 seconds to about 1 seconds, about 0.2 seconds to about 1 seconds, about 0.3 seconds to about 1 seconds, about 0.4 seconds to about 1 seconds, about 0.5 seconds to about 1 seconds, about 0.01 seconds to about 0.5 seconds, about 0.05 seconds to about 0.5 seconds, about 0.1 seconds to about 0.5 seconds, about 0.2 seconds to about 0.5 seconds, about 0.3 seconds to about 0.5 seconds, about 0.4 seconds to about 0.5 seconds, about 0.01 seconds to about 0.4 seconds, about 0.05 seconds to about 0.4 seconds, about 0.1 seconds to about 0.4 seconds, about 0.2 seconds to about 0.4 seconds, about 0.3 seconds to about 0.4 seconds, about 0.01 seconds to about 0.3 seconds, about 0.05 seconds to about 0.3 seconds, about 0.1 seconds to about 0.3 seconds, about 0.2 seconds to about 0.3 seconds, about 0.01 seconds to about 0.2 seconds, about 0.05 seconds to about 0.2 seconds, about 0.1 seconds to about 0.2 seconds, about 0.01 seconds to about 0.1 seconds, about 0.05 seconds to about 0.1 seconds, about 0.01 seconds to about 0.05 seconds, preferably about 3 seconds to about 5 seconds, more preferably about 4 seconds.

In some embodiments, the magnetically susceptible beads are washed with a washing solution and/or air. In some embodiments, the magnetically susceptible beads are washed sequentially and separately using both a washing solution and air. In some embodiments, the magnetically susceptible beads are alternately washed at least twice, at least three times, at least four times or at least five times with a washing solution and air, preferably wherein the magnetically susceptible beads are alternately washed twice with a washing solution and air.

In some embodiments, the biological sample is diluted before combining with the magnetically susceptible beads. In some embodiments, the electrode is a carbon ink electrode.

In some embodiments, the analyte of interest is brain natriuretic peptide or N-terminal pro-BNP. In some embodiments, the analyte of interest is cardiac troponin or cardiac troponin subunit I (cTnI).

In some embodiments, the one or more analytes of interest are selected from the list consisting of: brain natriuretic peptide, N-terminal pro-BNP, cardiac troponin and cardiac troponin subunit I (cTnI). In some embodiments, the one or more analytes of interest are N-terminal pro BNP and cardiac troponin subunit I (cTnI).

In some embodiments, the enzyme is horseradish peroxidase (HRP). In some embodiments, the enzyme is alkaline phosphatase (ALP). In some embodiments, at least one of the one or more second antibodies or antigen binding portions thereof is conjugated to horseradish peroxidase (HRP) and at least one of the one or more second antibodies or antigen binding portions thereof is conjugated to alkaline phosphatase (ALP).

In some embodiments, the substrate for the enzyme is selected from the list consisting of: 3,3′,5,5′-Tetramethylbenzidine (TMB), 2,2′-Azinobis [3-ethylbenzothiazoline-6-sulfonic acid]-diammonium salt (ABTS), o-phenylenediamine dihydrochloride (OPD), para-nitrophenyl phosphate (PNPP) and BCIP/NBT (a combination of BCIP (5-Bromo-4-chloro-3-indolyl phosphate) and NBT (nitro blue tetrazolium)).

In some embodiments, the product of the enzymatic reaction is precipitated, optionally wherein the product is precipitated onto the magnetically susceptible beads. In some embodiments, the substrate is 3,3′,5,5′-Tetramethylbenzidine (TMB) and the product of the enzymatic reaction is precipitated, optionally wherein the product is precipitated onto the magnetically susceptible beads. In some embodiments, the substrate is BCIP/NBT (a combination of BCIP (5-Bromo-4-chloro-3-indolyl phosphate) and NBT (nitro blue tetrazolium)) and the product of the enzymatic reaction is precipitated, optionally wherein the product is precipitated onto the magnetically susceptible beads.

In some embodiments, the electrochemical measurement is indicative of the concentration or amount of the analyte of interest. In some embodiments, the concentration or amount of the analyte of interest is determined by comparison to a reference solution.

In some embodiments, the electrochemical measurement is an amperometric, voltametric, potentiometric, impedimetric, or electrochemical impedance spectroscopic measurement, preferably a chronoamperometric measurement. In some embodiments, the electrochemical measurement is differential pulse voltammetry (DPV).

In some embodiments, the method of measuring is a sandwich immunoassay. In some embodiments, at least 50% (for example, at least 60%, at least 70%, at least 80%, at least 90% or at least 95% of the beads) measured by % weight are retained at the surface of the electrode.

The present invention also provides a method for measuring an analyte of interest in a biological sample comprising:
combining said biological sample with a composition comprising:
magnetically susceptible beads conjugated to a first antibody or antigen binding portion thereof capable of binding said analyte of interest; and
a second antibody or antigen binding portion thereof capable of binding said analyte of interest conjugated to an enzyme;
retaining the magnetically susceptible beads on an electrode using a magnetic field;
contacting the magnetically susceptible beads with a substrate for the enzyme, wherein the enzyme substrate is converted by the enzyme into a precipitated electroactive molecule; and
obtaining an electrochemical measurement using said electrode.

The present invention also provides a method for measuring an analyte of interest in a biological sample comprising:
combining said biological sample with a composition comprising:
magnetically susceptible beads conjugated to one or more antibodies or antigen binding portions thereof, wherein each antibody or antigen binding portion thereof is capable of binding one of said analytes of interest; and
one or more second antibodies or antigen binding portions thereof conjugated to an enzyme, wherein each second antibody or antigen binding portion thereof is capable of binding one of said analytes of interest;
retaining the magnetically susceptible beads on an electrode using a magnetic field;
contacting the magnetically susceptible beads with one or more substrates for the enzymes, wherein the enzyme substrates are converted by the enzymes into precipitated electroactive molecules; and
obtaining an electrochemical measurement using said electrode.

In some embodiments, the method is capable of detecting at least 2, 3, 4, 5, 6, 7, 8, 9 or 10 different analytes of interest. In some embodiments, the electroactive molecule is precipitated onto the magnetically susceptible beads. In some embodiments, the one or more analytes of interest are N-terminal pro BNP and cardiac troponin subunit I (cTnI). In some embodiments, the enzyme is horseradish peroxidase (HRP). In some embodiments, the enzyme is alkaline phosphatase (ALP). In some embodiments, at least one of the one or more second antibodies or antigen binding portions thereof is conjugated to horseradish peroxidase (HRP) and at least one of the one or more second antibodies or antigen binding portions thereof is conjugated to alkaline phosphatase (ALP).

In some embodiments, the substrate for the enzyme is selected from the list consisting of: 3,3′,5,5′-Tetramethylbenzidine (TMB), 2,2′-Azinobis [3-ethylbenzothiazoline-6-sulfonic acid]-diammonium salt (ABTS), o-phenylenediamine dihydrochloride (OPD), para-nitrophenyl phosphate (PNPP) and BCIP/NBT (a combination of BCIP (5-Bromo-4-chloro-3-indolyl phosphate) and NBT (nitro blue tetrazolium)).

In some embodiments, the magnetically susceptible beads and the biological sample are incubated at a temperature of between 10° C. and 50° C., optionally between 15° C. and 45° C., further optionally between 20° C. and 40° C., further optionally between 20° C. and 30° C., further optionally between 25° C. and 35° C., further optionally at 25° C., further optionally at 30° C., further optionally at 40° C.

The present invention also provides a composition comprising magnetically susceptible beads conjugated to a first antibody or antigen binding portion thereof capable of binding to an analyte of interest; and
a second antibody or antigen binding portion thereof capable of binding said analyte of interest conjugated to an enzyme.

The present invention also provides a composition comprising magnetically susceptible beads conjugated to one or more antibodies or antigen binding portions thereof, wherein each antibody or antigen binding portion thereof is capable of binding an analyte of interest; and
one or more second antibodies or antigen binding portions thereof conjugated to an enzyme, wherein each second antibody or antigen binding portion thereof is capable of binding one of said analytes of interest.

In some embodiments of compositions of the invention, each magnetically susceptible bead is conjugated to multiple different antibodies or antigen binding portions thereof, wherein each antibody or antigen binding portion thereof is capable of binding one of said analytes of interest. In some embodiments of compositions of the invention, the magnetically susceptible beads comprise different sets of magnetically susceptible beads, wherein each set of magnetically susceptible beads is conjugated to a different antibody or antigen binding portion thereof, wherein each antibody or antigen binding portion thereof is capable of binding one of said analytes of interest.

In some embodiments of compositions of the invention, the composition is capable of detecting at least 2, 3, 4, 5, 6, 7, 8, 9 or 10 different analytes of interest. In some embodiments of compositions of the invention, the analyte of interest is brain natriuretic peptide or N terminal pro-BNP. In some embodiments of compositions of the invention, the analyte of interest is cardiac troponin or cardiac troponin subunit I (cTnI). In some embodiments of compositions of the invention, the one or more analytes of interest are N-terminal pro BNP and cardiac troponin subunit I (cTnI). In some embodiments of compositions of the invention, the enzyme is horseradish peroxidase (HRP) or alkaline phosphatase (ALP). In some embodiments of compositions of the invention, at least one of the one or more second antibodies or antigen binding portions thereof is conjugated to horseradish peroxidase (HRP) and at least one of the one or more second antibodies or antigen binding portions thereof is conjugated to alkaline phosphatase (ALP).

In some embodiments of compositions of the invention, the substrate for the enzyme is selected from the list consisting of: 3,3′,5,5′-Tetramethylbenzidine (TMB), 2,2′-Azinobis [3-ethylbenzothiazoline-6-sulfonic acid]-diammonium salt (ABTS), o-phenylenediamine dihydrochloride (OPD), para-nitrophenyl phosphate (PNPP) and BCIP/NBT (a combination of BCIP (5-Bromo-4-chloro-3-indolyl phosphate) and NBT (nitro blue tetrazolium)).

The present invention also provides a kit for performing a method according to the invention, comprising:
magnetically susceptible beads;
an immunoassay apparatus comprising an electrode; and
a magnet positioned proximate to the chip for retaining the magnetically susceptible beads proximate to the electrode.

In some embodiments of kits of the invention, the kit further comprises a means of retaining the magnetically susceptible beads at a separate location spatially distant from the electrode. In some embodiments of kits of the invention, the means of retaining the magnetically susceptible beads at a separate location spatially distant from the electrode is the same magnet used to retain the magnetically susceptible beads proximate to the electrode. In some embodiments of kits of the invention, the means of retaining the magnetically susceptible beads at a separate location spatially distant from the electrode is a second magnet configured to retain the magnetically susceptible beads at a separate location spatially distant from the electrode. In some embodiments of kits of the invention, the magnet is a permanent magnet or an electromagnet.

In some embodiments of kits of the invention, the kit further comprises a means of retaining the magnetically susceptible beads at a separate location spatially distant from the electrode. In some embodiments of kits of the invention, the means of retaining the magnetically susceptible beads at a separate location spatially distant from the electrode is the same magnet used to retain the magnetically susceptible beads proximate to the electrode. In some embodiments of kits of the invention, the means of retaining the magnetically susceptible beads at a separate location spatially distant from the electrode is a second magnet configured to retain the magnetically susceptible beads at a separate location spatially distant from the electrode. In some embodiments of kits of the invention, the magnet is a permanent magnet or an electromagnet.

The present invention also provides a method for measuring an analyte of interest in a biological sample comprising:
combining said biological sample with a composition comprising:
   magnetically susceptible beads conjugated to a first antibody or antigen binding portion thereof capable of binding said analyte of interest; and
   a second antibody or antigen binding portion thereof capable of binding said analyte of interest conjugated to an enzyme;
retaining the magnetically susceptible beads on an electrode using a magnetic field;
contacting the magnetically susceptible beads with a substrate for the enzyme, wherein the enzyme substrate is converted by the enzyme into a precipitated electroactive molecule; and
obtaining an electrochemical measurement using said electrode, wherein the biological sample and the composition are combined at a position spatially distant from said electrode, optionally wherein the spatially distant position is a blank or non-functional electrode.

The present invention also provides a method for measuring an analyte of interest in a biological sample comprising:
combining said biological sample with a composition comprising:
   magnetically susceptible beads conjugated to one or more antibodies or antigen binding portions thereof, wherein each antibody or antigen binding portion thereof is capable of binding one of said analytes of interest; and
   one or more second antibodies or antigen binding portions thereof conjugated to an enzyme, wherein each second antibody or antigen binding portion thereof is capable of binding one of said analytes of interest;
retaining the magnetically susceptible beads on an electrode using a magnetic field;
contacting the magnetically susceptible beads with one or more substrates for the enzymes, wherein the enzyme substrates are converted by the enzymes into precipitated electroactive molecules; and
obtaining an electrochemical measurement using said electrode, wherein the biological sample and the composition are combined at a position spatially distant from said electrode, optionally wherein the spatially distant position is a blank or non-functional electrode.

In some embodiments, the magnetically susceptible beads are contacted with one or more substrates for the enzymes at a position spatially distant from said electrode. In some embodiments, the magnetically susceptible beads are washed at a position spatially distant from said electrode with a washing solution and/or air. In some embodiments, the magnetically susceptible beads are washed at a position spatially distant from said electrode sequentially and separately using both a washing solution and air.

In some embodiments, the magnetically susceptible beads are alternately washed at a position spatially distant from said electrode at least twice, at least three times, at least four times or at least five times with a washing solution and air, preferably wherein the magnetically susceptible beads are alternately washed at a position spatially distant from said electrode twice with a washing solution and air. In some embodiments, the biological sample and the composition are combined at a position spatially distant from said electrode and then the magnetically susceptible beads are moved to said electrode to obtain an electrochemical measurement. In some embodiments, the magnetically susceptible beads are contacted with one or more substrates for the enzymes at a position spatially distant from said electrode and then the magnetically susceptible beads are moved to said electrode to obtain an electrochemical measurement.

In some embodiments, the magnetically susceptible beads are contacted with one or more substrates for the enzymes at a position spatially distant from said electrode and then the magnetically susceptible beads are moved to said electrode to obtain an electrochemical measurement.

In some embodiments, the biological sample and the composition are combined at a position spatially distant from said electrode; and/or the magnetically susceptible beads are retained and washed at a position spatially distant from said electrode with a washing solution and/or air and then the magnetically susceptible beads are moved to said electrode to obtain an electrochemical measurement, optionally wherein the spatially distant position for the combining of the biological sample and the composition and the spatially distant position for the washing are the same.

In some embodiments, the biological sample and the composition are combined at a position spatially distant from said electrode, the magnetically susceptible beads are contacted with one or more substrates for the enzymes at a position spatially distant from said electrode; and/or the magnetically susceptible beads are retained and washed at a position spatially distant from said electrode with a washing solution and/or air and then the magnetically susceptible beads are moved to said electrode to obtain an electrochemical measurement, optionally wherein the spatially distant position for the combining of the biological sample and the composition, the spatially distant position for the washing and/or the spatially distant position for contacting the magnetically susceptible beads with one or more substrates for the enzymes are the same.

In some embodiments of the invention the first antibody or antigen binding portion thereof capable of binding said analyte of interest binds to the same portion or epitope of the analyte of interest as the second antibody or antigen binding portion thereof capable of binding said analyte of interest. In preferred embodiments of the invention the first antibody or antigen binding portion thereof capable of binding said analyte of interest binds to a first portion or epitope of the analyte of interest and the second antibody or antigen binding portion thereof capable of binding said analyte of interest binds to a second portion or epitope of the analyte of interest. In some embodiments, first portion or epitope of the analyte of interest is distant from the second portion or epitope of the analyte of interest, thereby reducing steric hinderance of having two antibodies or antigen binding portions thereof binding to the same analyte of interest.

### BRIEF DESCRIPTION OF THE DRAWINGS

Embodiments of the present invention will now be described, by way of example only, with reference to the accompanying drawings, in which:

FIG. 1—A. Signal comparison with or without actuation of the magnet after pulling down on the electrode. B. Visualisation of beads spread onto the electrode with or without actuation of the magnet after pulling down the magnetic particle.

FIG. 2—Assessment of introduction of actuation method during the wash process for beads assay (chronoamperometry current after 60 seconds) for samples containing 0 ng/L analyte.

FIG. 3—Assessment of possible reduction of wash volumes when using magnet actuation method during the wash process for beads assay (chronoamperometry current after 60 seconds) between samples containing zero and 100 pg/ml analyte.

FIG. 4—Signal to noise improvement using magnet actuation method (chronoamperometry current after 60 seconds) between samples containing zero and 50 ng/L analyte.

FIG. 5—Bead resuspensions using air-liquid interfaces (chronoamperometry current after 60 seconds) for samples containing 100 ng/L analyte.

FIG. 6—Differential pulse voltammetry (DPV) measurements for 0 and 50 ng/ml concentration of troponin in troponin free serum. Peak high nA was recorded for N=16 for each group.

FIG. 7—Plotting of signal-to-noise (S/N) ratio for the two concentrations 0 and 50 ng/ml of troponin. N=16 per group.

FIG. 8—Isometric view of an exemplary liquid handling device.

FIG. 9—Exploded view showing the components of the liquid handling device in FIG. 8.

FIG. 10—Top sectional view through the liquid handling device shown in FIG. 8.

FIG. 11—An isometric underside view of a first rigid layer of the liquid handling device shown in FIG. 8.

FIG. 12—Top view of a fluidic layer that may be implemented in the liquid handling device shown in FIG. 8.

FIG. 13—Bottom view of the alternative fluidic layer shown in FIG. 12.

### DETAILED DESCRIPTION OF THE INVENTION

### Suitable Microfluidic Devices

According to a first aspect of the present disclosure, the methods of the present invention can be carried out on a liquid handling device, comprising: a first rigid layer and a second rigid layer; a fluidic layer disposed between the first rigid layer and the second rigid layer; wherein the fluidic layer is formed of an elastomer; and wherein the fluidic layer comprises a network of channels; and a fluidic network comprising a plurality of conduits, wherein the plurality of conduits are defined at least in part by the network of channels in the fluidic layer.

Using channels provided in an elastomeric layer provides improved sealing of the fluidic network, irrespective of the bonding process used to seal the network (e.g. pressure-sensitive adhesive (PSA) tape, laser welding, etc.). This is because the elastomer layer acts as a compliant layer when it is being sealed against another layer. In addition, providing channels in a compliant elastomeric layer allows the channels to be compressed in order to provide valves in the liquid handling device. Liquid flows in the liquid handling device can therefore be controlled by compressing the channels in the elastomeric layer, which closes valves of the liquid handling device. Using a single layer for the network of channels also simplifies the construction of the liquid handling device.

The liquid handling device may further comprise a plurality of valves. Each of the plurality of valves may be configured to close a corresponding one of the plurality of conduits. The valves allow liquid flow within the liquid handling device to be controlled.

Each of the plurality of valves may comprise a deformable valve region provided in the fluidic layer. Each deformable valve region may be deformable to a deformed state in which the corresponding one of the plurality of conduits is blocked. Providing deformable valve regions in the fluidic layer simplifies the construction of the liquid handling device, because the fluidic layer implements both the conduits of the device and the valves of the device.

The fluidic layer may comprise a first face configured to face the first rigid layer and a second face configured to face the second rigid layer. At least part of the network of channels may be provided in the second face. Each deformable valve region may comprise a depression in the first face of the fluidic layer. The depression may be aligned with a corresponding channel of the at least part of the network of channels provided in the second face. Providing depressions in the first face of the fluidic layer reduces the volume of material that needs to be deformed in order to close each valve of the liquid handling device. This reduces the force required to close each of the valves.

A subset of the network of channels may be provided in the first face. Providing channels in both faces of the fluidic layer means increases the available area for providing the channels with respective bonding areas around them, which is particularly important in view of the limited real estate available on the fluidic layer, resulting from the small size of point-of-care devices. Providing channels in the first face also allows the channels of the fluidic layer to cross over, meaning that more complex networks of channels may be implemented.

The first rigid layer may comprise a plurality of apertures. Each deformable valve region may be accessible through one of the plurality of apertures. Providing apertures in the rigid layer means that the liquid handling device has a rigid housing, while allowing the valves to be actuated by application of an external force (e.g. from an actuator of an analyser device).

The liquid handling device may further comprise a plurality of openings extending through at least part of the thickness of the fluidic layer. Each of the plurality of openings may be in fluidic communication with one of the plurality of conduits. The plurality of openings may comprise a first plurality of openings and a second plurality of openings. The second plurality of openings may be different to the first plurality of openings. The plurality of openings allows fluid (i.e. either liquid, or air supplied from a pneumatic supply system) in the fluidic layer to communicate with fluidic components in other layers.

The liquid handling device may further comprise a plurality of ports configured to provide a seal against a pneumatic interface. Each of the plurality of ports may comprise: a protrusion protruding from a surface of the fluidic layer, and a respective one of the first plurality of openings. The respective one of the first plurality of openings may extend through the protrusion. Implementing ports in an elastomeric fluidic layer allows the ports to form a seal with a pneumatic interface. This is because the fluidic layer acts as a compliant layer when a force is applied to the port by a pneumatic interface (e.g. a pneumatic actuator of a pneumatic supply system). Providing ports in the same fluidic layer as the network of channels also simplifies the construction of the liquid handling device. The fluidic communication between the ports and the conduits allows liquid to be moved within the conduits, by applying pneumatic pressures via the ports.

Each protrusion may have a frustoconical shape. The frustoconical shape of the protrusions helps formation of the seal between the port and a pneumatic interface. This is because the frustoconical shape results in a narrowing of the cross-section of the protrusion, with increasing height above the surface. Put another way, the frustoconical shape results in less material at the top of the protrusion than at the base of the protrusion, owing to the angled walls provided by the frustoconical shape. The reduced cross-section at the top of the protrusion means that less material is required to be deformed by a pneumatic interface, in order to provide a seal around the port. Deforming less material means that a lower amount of force needs to be applied to compress the port.

Each of the first plurality of openings may have a diameter that increases with increasing height above the surface of the fluidic layer. This further reduces the amount of material at the top of the protrusion, resulting in a lower force being required to deform the port.

Each protrusion may comprise an annular rim around an open end of the protrusion. The annular rim may define a region of minimum cross-sectional area of the protrusion. The annular rim provides a further reduction in the amount of material at the top of the protrusion, meaning that the force required to deform the protrusion is reduced.

One or more of the plurality of ports may further comprise: a plurality of support ribs. Each of the plurality of support ribs may extend between the protrusion and the surface of the fluidic layer from which the protrusion protrudes. The support ribs help to prevent excessive deformation of the ports when forces are applied to the ports by a pneumatic interface.

The first rigid layer may comprise a plurality of apertures. Each port may be accessible through one of the plurality of apertures. Providing apertures in the rigid layer means that the liquid handling device has a rigid housing, while allowing pneumatic pressure to be applied to the ports using an external pneumatic interface (e.g. a pneumatic actuator of an analyser device).

Each of the plurality of ports may be in fluidic communication with one of the plurality of conduits via a corresponding trough in the second rigid layer. The troughs prevent liquid from reaching the ports, which connect to pneumatic interfaces. Accordingly, the troughs prevent liquid from reaching the pneumatic interfaces, particularly during aspiration of liquid. Such liquid could potentially contaminate or damage the pneumatic interfaces (e.g. in an analyser device). In particular, any liquid drawn from the channels in the fluidic layer during aspiration pools in the bottom of the trough and does not reach the port. Therefore, any liquid drawn from the channels is not drawn into the pneumatic interface via the port.

The liquid handling device may further comprise at least one liquid storage capsule disposed over two of the second plurality of openings. Disposing a liquid storage capsule over the openings allows the fluidic network to interface with the liquid storage capsule. This also allows the capsule to be deformed into the openings, to create openings in the capsule.

The fluidic layer may comprise one or more chambers. Each of the one or more chambers is in fluidic communication with one of the plurality of conduits. Providing a chamber in the fluidic layer results in a simple construction of the liquid handling device. In particular, providing the one or more chambers in the fluidic layer extends the functionality of the fluidic layer.

The fluidic layer may comprise a projection extending from a face of the fluidic layer. The projection may comprise a plurality of cavities. Each of the one or more chambers may be defined at least in part by a corresponding one of the plurality of cavities. Providing a projection that extends from a face of the fluidic layer means that the volume of the chamber is not limited by the thickness of the fluidic layer. An increased chamber capacity can therefore be provided.

The liquid handling device may further comprise a sealing film. The plurality of conduits may be defined by the network of channels in the fluidic layer and the sealing film. The compliance of the elastomeric fluidic layer helps the channels to be sealed by the sealing film.

Each channel comprises a groove provided in a surface. Each channel therefore has an open cross-section. In other words, the cross-section of each channel is not sealed. Each conduit comprises: (i) a channel that is sealed (e.g. by a sealing layer), thereby providing a closed cross-section, or (ii) a hole or tunnel extending at least partially through a body.

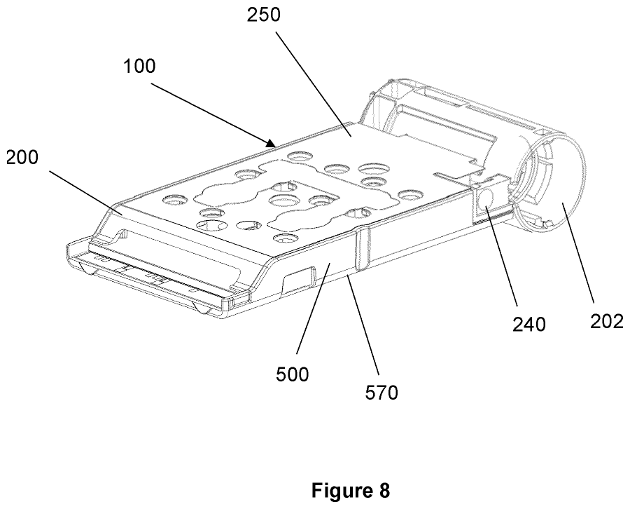

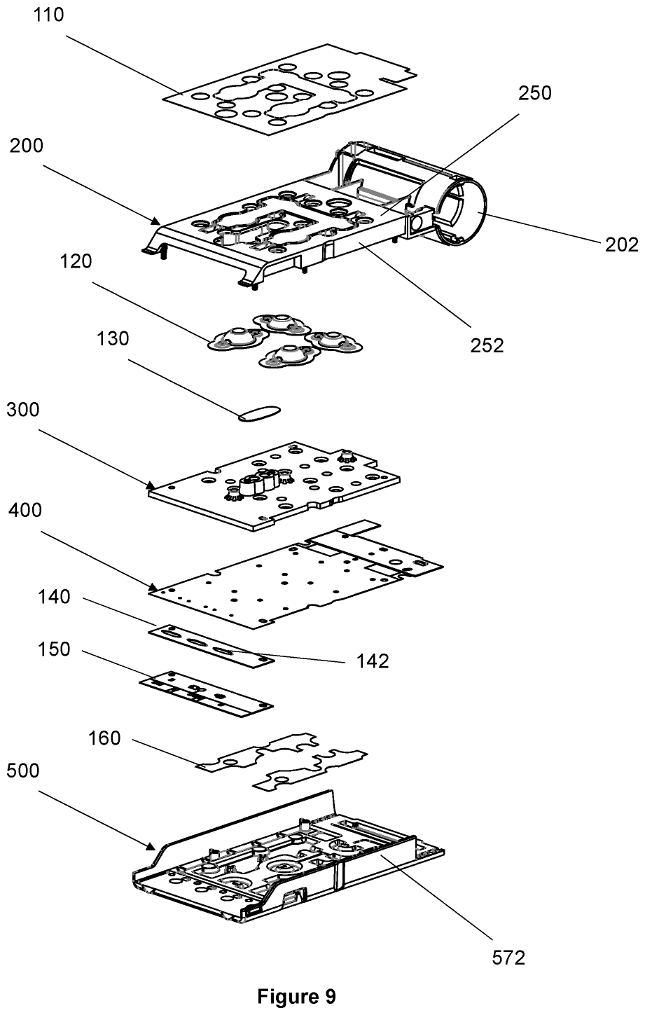

FIG. 8 is an isometric view of a liquid handling device in the form of a diagnostic cartridge 100 (e.g. a microfluidic cartridge). The cartridge 100 comprises a number of components, as seen from the exploded view shown in FIG. 9.

Specifically, the cartridge 100 comprises a first part 200 and a second part 500, each of which is formed of a rigid material. In use (i.e. when the cartridge 100 is in the orientation shown in FIG. 8), the first part 200 is an upper part and the second part 500 is a lower part. Together, the first part 200 and the second part 500 define a housing of the cartridge 100. Specifically, the first part 200 comprises a rigid face 250 that defines an upper surface of the cartridge 100. Likewise, the second part 500 comprises a rigid face 570 (as best shown in FIG. 8) that defines a lower surface of the cartridge 100. Returning to FIG. 9, it can be seen that the first part 200 further comprises side walls 252 that are joined to the rigid face 250, while the second part 500 further comprises side walls 572 joined to the rigid face 570. Together, the side walls 252 of the first part 200 and the side walls 572 of the second part 500 cooperate to define the side walls of the cartridge 100.

The cartridge 100 further comprises a fluidic layer 300 disposed within the housing defined by the first part 200 and the second part 500. Specifically, the fluidic layer 300 is disposed between the rigid face 250 of the first part 200 and the rigid face 570 of the second part 500. Therefore, the fluidic layer 300 is disposed between a first rigid layer in the form of the rigid face 250, and a second rigid layer in the form of the rigid face 570. The fluidic layer 300 is formed of an elastomeric material, such as a thermoplastic elastomer (TPE), for example, a silicon-based TPE or styrene-ethylene-butylene-styrene (SEBS); polydimethylsiloxane (PDMS); or liquid silicone rubber (LSR).

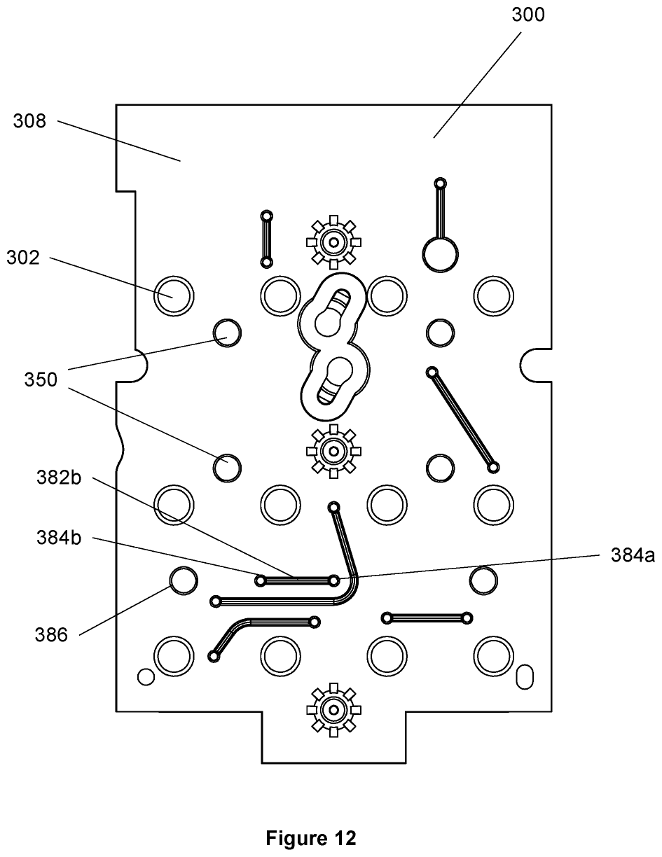

As described in more detail below, with reference to FIG. 12, a first surface 308 of the fluidic layer 300 comprises a plurality of valve regions 302. The cartridge 100 is received in an analyser device that comprises actuators that apply forces to the valve regions 302 of the fluidic layer 300, to close one or more conduits within the cartridge 100. The properties of the material used for the fluidic layer 300 are dependent on the available force that can be applied to the valve regions 302 of the fluidic layer 300 by the actuators. Two properties of importance are the hardness of the material, and the relaxation time of the material (i.e. the time for the material to return to its original form following deformation). Examples of suitable materials include the elastomeric materials listed above. In some implementations, the fluidic layer 300 may be a medical-grade material, to prevent reaction of the fluidic layer 300 with the reagents used in the diagnostic test or assay.

As described in more detail below, the fluidic layer 300 comprises a network of channels 304 provided (at least partly) in a second surface 310 of the fluidic layer 300 that is opposite to the first surface 308. The cartridge 100 also comprises a fluidic network comprising a plurality of conduits, which are defined at least in part by the network of channels 304 in the fluidic layer 300. Specifically, the conduits are defined by: (i) the network of channels 304 in the fluidic layer 300; (ii) a sealing layer 400 (shown in FIG. 9) that is configured to seal the channels 304 in the second surface 310 of the fluidic layer 300; and optionally (iii) a sealing layer (not shown) configured to seal any channels 304 of the network that are provided in the first surface 308.

Providing channels 304 in an elastomeric fluidic layer 300 provides improved sealing of the fluidic layer, irrespective of the bonding process used to seal the network of channels 304 (e.g. pressure-sensitive adhesive tape, laser welding, etc.). This is because the elastomeric fluidic layer 300 acts as a compliant layer when it is being sealed against another layer (e.g. sealing layer 400). In addition, using an elastomeric material for the fluidic layer 300 means that the channels 304 can be compressed in order to close respective conduits. This means that a single layer can be utilised to implement the channels 304 and valves (i.e. valve regions 302), thereby providing a simple cartridge construction.

Returning to the exploded view shown in FIG. 9, it can be seen that the cartridge 100 further comprises: a label 110 arranged to cover at least a portion of the rigid face 250 of the first part 200; a plurality of liquid storage capsules 120 that are disposed within the cartridge 100 between the fluidic layer 300 and the first face 250; and a sealing tape 130 arranged to seal one or more chambers 332 in the fluidic layer 300.

FIG. 9 also shows that the cartridge 100 further comprises: a flow cell strip 140 comprising a plurality of apertures 142, each of which defines, in part, a corresponding measurement chamber 610 of the cartridge 100; a sensor strip 150 comprising a plurality of sensors, each sensor in fluidic communication with a respective one or the measurement chambers 610; and a pair of absorbent waste pads 160, each arranged to fit within a corresponding waste chamber provided in the second part 500. In some implementations, the flow cell strip 140 is not present, and apertures 142 that define in part the measurement chambers 610 are instead provided in an alternative sealing layer.

As shown in FIGS. 8 and 9, the first part 200 comprises a receptacle in the form of a cylinder 202, which is configured to receive a portion of a liquid storage container such as a blood collection tube (e.g. a Vacutainer® blood collection tube manufactured by Becton, Dickinson and Company of Franklin Lakes, NJ, USA). A blood collection tube typically contains a volume of liquid (e.g. blood), and a headspace that includes a volume of gas.

The assay methods disclosed are not limited to biological samples derived from a particular source. The assay methods disclosed can also be implemented from capillary blood samples, plasma samples or any other suitable receptacle containing a biological sample, such as a blood sample, from a subject.

### Fluidic Layer

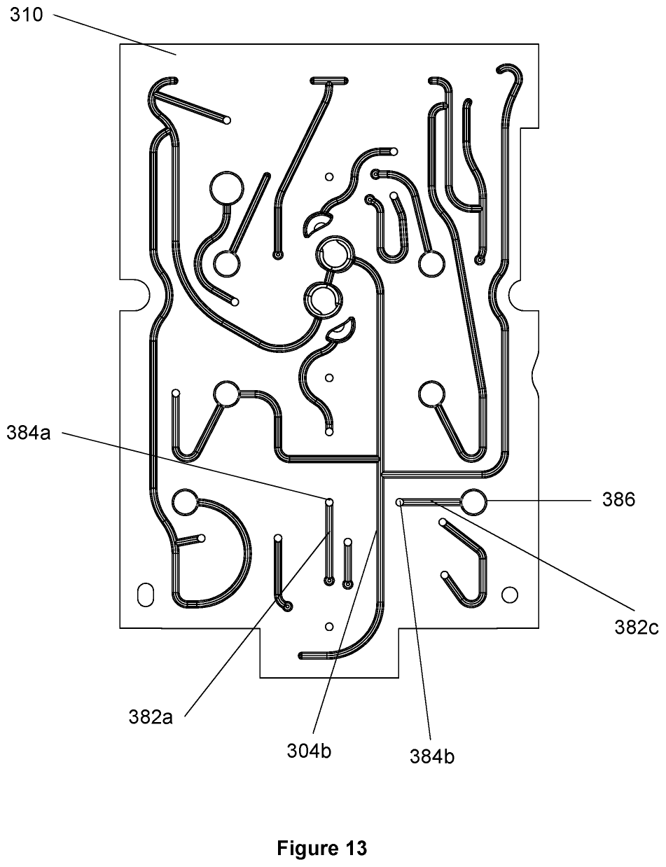

In an implementation of the fluidic layer, shown in FIGS. 12 and 13, channels 304 are provided in both a first surface 308 and a second surface 310 of a fluidic layer 300. Configuring a network of fluidic channels in a limited amount of space is challenging. Point-of-care devices are designed to be small, which limits the real estate available on the fluidic layer 300 for laying down the channels with respective bonding areas around them (i.e. for bonding to the sealing layer 400). Implementing channels 304 on both surfaces 308, 310 of the fluidic layer 300 allows, for example, channels 304 used for transporting air (e.g. for clearing the measurement chambers, or for displacing liquid from liquid storage capsules 120) to be moved to the first surface 308, without affecting liquid flow. Providing channels 364 in the first surface 308 also allows the channels 304 of the fluidic layer 300 to cross over, meaning that more complex networks of channels 304 may be implemented.

In the implementation of the fluidic layer 300, valve regions 302 are still provided in the first surface 308 of the fluidic layer 300. Accordingly, the channels 304 in the first surface 308 are either channels 304 that do not pass under a valve region 302, or channels 304 that have a first portion in the first surface 308 and a second portion in the second surface 310. For example, the second portion of the channel 304 may be a portion of the channel 304 that passes under a valve region 302. The two portions of these channels 304 may be connected by vertical or angled conduits running through the thickness of the fluidic layer 300.

In FIGS. 12 and 13, various examples of channels 304 with portions in both surfaces 308, 310 are shown. For example, channel 304a in FIGS. 12 and 13 includes a first portion 382a provided in the second surface 310, a second portion 382b provided in the first surface 308, and a third portion 382c provided in the second surface 310. The first portion 382a extends between a point overlying the first trough 514a (shown in FIG. 9) to a first through-hole 384a in the fluidic layer 300. The second portion 382b extends between the first though-hole 384a and a second through-hole 384b in the fluidic layer. The third portion 382c extends between the second through-hole 384b and an opening 386 over which a liquid storage capsule 120 may be disposed, when a cartridge 100 comprising the fluidic layer 300 is assembled. By providing portions of the channel 304a in both surfaces 308, 310, the channel 304b can cross over the channel 304a (as shown in FIGS. 12 and 13).

### Liquid Storage Capsules

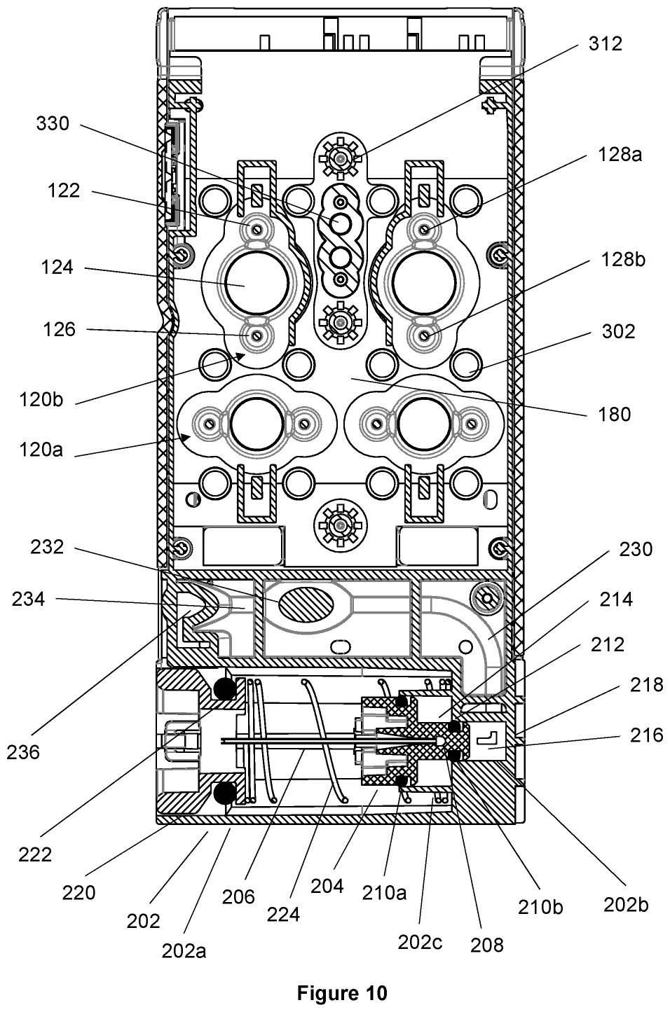

FIG. 10 also shows the arrangement of the plurality of liquid storage capsules 120 within the cartridge 100. In particular, the liquid storage capsules 120 are sealed to the fluidic layer 300 using a sealing tape 180. FIG. 10 shows that the sealing tape 180 includes apertures that allow the upwardly protruding features of the fluidic layer 300 (i.e. pneumatic ports 312 and a projection 330 that defines the chambers 332. As shown in FIG. 10, each liquid storage capsule 120 comprises an inlet chamber 122, a main chamber 124 storing a liquid such as a liquid reagent, and an outlet chamber 126. A sealing layer (e.g. a sealing foil) is used to seal the chambers 122, 124, 126 of each liquid storage capsule 120. The inlet chamber 122 and the outlet chamber 126 each comprise a corresponding recess 128a, 128b in a top surface of the chamber.

The liquid storage capsules 120 shown in FIG. 10 comprise two smaller liquid storage capsules 120a, and two larger liquid storage capsules 120b. The smaller liquid storage capsules 120a are aligned such that the recesses 128a, 128b of the smaller storage capsules 120a are all in a straight line. Each of the larger liquid storage capsules 120b is arranged perpendicular to a corresponding smaller liquid storage capsule 120a, such that the larger liquid storage capsules 120b are parallel to each other.

As explained in more detail below, each of the liquid storage capsules 120 is positioned over two openings 350 in the fluidic layer 300. Specifically, the inlet chamber 122 of a liquid storage capsule 120 covers a first one of the openings 350, while the outlet chamber 126 of the liquid storage capsule 120 covers a second one of the openings 350. When forces are applied to the recesses 128a, 128b of the liquid storage capsule 120, the material of the liquid storage capsule 120 is deformed into each of the openings 350. When sufficient force is applied, the deformation of the liquid storage capsule 120 into the openings 350 causes rupture of the sealing layer (e.g. foil) used to seal the capsule 120.

In an alternative implementation, the inlet chamber 122 and the outlet chamber 126 may not comprise recesses 128. Instead, forces may be applied directly to a portion of the inlet chamber 122 and the outlet chamber 126 to deform the liquid storage capsule 120.

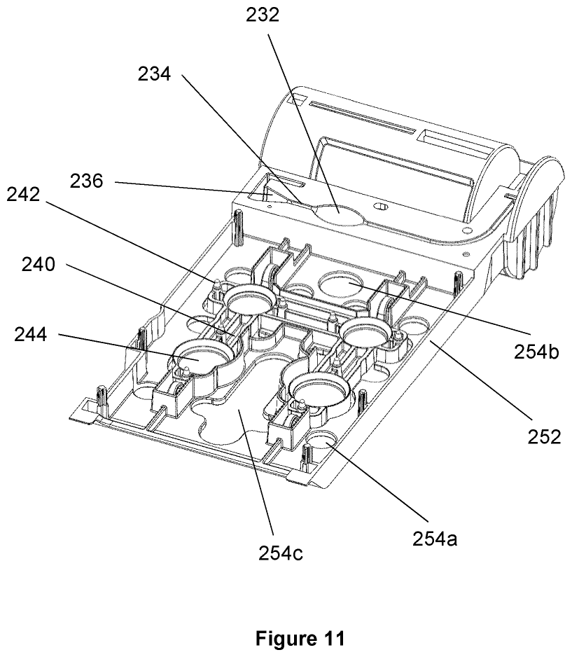

FIG. 11 is an isometric underside view of the first part 200. As shown in FIG. 11, the first part 200 comprises an actuatable portion 240 (e.g. an actuatable platform) that is actuatable from a first position, in which the actuatable portion 240 does not deform the liquid storage capsules 120, to a second position, in which the actuatable portion 240 deforms the liquid storage capsules 120.

The actuatable portion 240 is U-shaped, such that it can be deformed towards each of the liquid storage capsules 120. The U-shape of the actuatable portion 240 also allows the actuatable portion 240 to pass around a projection 330 that extends from the first surface 308 of the fluidic layer 300.

As shown in FIG. 11, the underside of the actuatable portion 240 comprises four pairs of protrusions 242. Each pair of protrusions 242 extends towards the liquid storage capsules 120 and is aligned with the recesses 128a, 128b of one of the liquid storage capsules 120. Therefore, when the actuatable portion 240 is moved to the second position, the protrusions 242 engage the recesses 128a, 128b of the capsules 120. In an alternative implementation, the liquid storage capsules 120 may not include recesses 128a, 128b, in which case the protrusions 242 may engage a portion of the inlet and outlet chambers 122, 126 (e.g. a flat or domed upper surface of the inlet and outlet chambers 122, 126) of each liquid storage capsule 120.

The underside of the actuatable portion 240 also includes four concave regions 244. Each concave region 244 is located between two of the protrusions 242. Each concave region 244 is configured to accommodate the main chamber 124 of its corresponding liquid storage capsule 120 when the actuatable portion 240 is in the second position. This means that the main chamber 124 is not deformed by the actuatable portion 240 when the actuatable portion 240 is in the second position.

Given that the protrusions 242 extend from a single actuatable portion 240, actuation of the actuatable portion 242 to the second position causes simultaneous deformation of each of the plurality of capsules 120. Consequently, all capsules 120 within the cartridge 100 can be punctured using a single movement of the actuatable portion 240.

In an alternative implementation, the actuatable portion 240 may comprise two sets of protrusions 242: a first set of protrusions, each extending a first distance towards the recesses 128 of the liquid storage capsules 120; and a second set of protrusions, each extending a second distance towards the recesses 128 of the liquid storage capsules 120, wherein the second distance is less than the first distance.

This alternative implementation allows for puncture of the liquid storage capsules 120 in two stages. The capsules 120 aligned with the first set of protrusions are punctured first, when the actuatable portion 240 is moved to the second position (as described above). However, to puncture the capsules 120 aligned with the second set of protrusions, the actuatable portion 240 is actuated beyond the second position, to a third position (because the second set of protrusions are shorter). This alternative implementation therefore allows for liquid (e.g. liquid reagent) to be released from some capsules before other capsules are punctured.

Therefore, fluidic workflow steps involving, for example, liquid reagents stored in first and second capsules may be completed prior to release of liquid reagent from third and fourth capsules (e.g. if the liquid reagents in the third and fourth capsules are required at a later stage of the fluidic workflow). Additional sets of protrusions extending different distances from the actuatable portion 240 may be implemented in order to further stagger the release of liquids from the capsules 120.

Devices that Allow Bidirectional Flow

Immunoassays rely on delivery of liquids in a controlled manner. The volume of the liquid delivered and the time of interactions are critical to the success and reproducibility of the assay. In addition, heterogeneous immunoassays require wash steps, to remove unbound antibodies, unbound antigen and enzyme tags, from the detection surfaces. Reagents can be trapped in the liquid flow path and then interact in nonspecific reactions. This can increase the background signal which reduces the assay sensitivity, dynamic range and precision. Assay performance can be significantly improved by using different flow paths and/or different liquid flow directions to add reagents that can potentially cross-react.

Configurations of liquid handling devices which provide bi-directional flow allows rapid, precise and controllable quenching of reactions and/or biological interactions in the measurement chamber. The use of conduits with different flow directions also provides reduced contamination of each liquid during different method steps (i.e. reduced contamination of sample liquid in a wash step). This may not be readily achievable with known fluid handling devices, such as conventional microfluidic devices.

In one aspect a liquid handling device may comprise a sample chamber for receiving a sample; a measurement chamber for performing one or more measurements on the sample wherein the measurement chamber comprises a reaction zone; a first liquid reagent chamber; a sample chamber conduit which fluidically connects the sample chamber to the measurement chamber; a sample chamber conduit valve for opening and closing the sample chamber conduit; a first liquid reagent chamber conduit which fluidically connects the first liquid reagent chamber to the measurement chamber in an alternate flow direction to the sample chamber conduit; and a first liquid reagent chamber conduit valve for opening and closing the first liquid reagent chamber conduit.

The flow direction of the first liquid reagent chamber conduit into the measurement chamber may be at least ninety degrees to the flow direction of the sample chamber conduit into the measurement chamber. In one embodiment the flow direction of the first liquid reagent chamber conduit into the measurement chamber is opposite to the flow direction of the sample chamber conduit into the measurement chamber. In some embodiments opposite flow direction is equivalent to a second flow direction that is 180 degrees to a first flow direction in the same horizontal plane of the device.

In some embodiments the device further comprises a second liquid reagent chamber; a second liquid reagent chamber conduit which fluidically connects the second liquid reagent chamber to the measurement chamber in an alternate flow direction to the sample chamber conduit; and a second liquid reagent chamber conduit valve for opening and closing the second liquid reagent chamber conduit.

In some embodiments the second liquid reagent chamber conduit fluidically connects to the measurement chamber in an alternate direction to both the sample chamber conduit and the first liquid reagent chamber conduit.

In some embodiments the second liquid reagent chamber conduit is fluidically connected to the first liquid reagent chamber conduit thereby providing a combined conduit, fluidically connecting both the first liquid reagent chamber and second liquid reagent chamber to the measurement chamber. In some embodiments the flow direction of the combined conduit into the measurement chamber is at least ninety degrees to the flow direction of the sample chamber conduit into the measurement chamber.

In some embodiments the flow direction of the combined conduit into the measurement chamber is opposite to the flow direction of the sample chamber conduit into the measurement chamber. In some embodiments opposite flow direction is equivalent to a second flow direction that is 180 degrees to a first flow direction in the same horizontal plane of the device.

In some embodiments the flow direction of the second liquid reagent chamber conduit into the measurement chamber is at least ninety degrees to the flow direction of the sample chamber conduit and/or the first liquid chamber conduit into the measurement chamber.

In some embodiments the flow direction of the second liquid reagent chamber conduit into the measurement chamber is opposite to the flow direction of the sample chamber conduit and/or the first liquid chamber conduit into the measurement chamber. In some embodiments opposite flow direction is equivalent to a second flow direction that is 180 degrees to a first flow direction in the same horizontal plane of the device.

In some embodiments the reaction zone comprises one or more electrodes. In some embodiments the one or more electrodes comprise one or more electrodes selected from the list:
counter electrode, reference electrode and working electrode. In some embodiments the one or more electrodes comprise at least one working electrode.

In some embodiments the device comprises two or more measurement chambers, each of which is fluidically connected to the sample chamber and each of which is fluidically connected to the first liquid reagent chamber, wherein the device comprises a corresponding number of sample chamber conduit valves and/or first liquid reagent chamber valves for independent control of the flow of sample liquid and/or first liquid reagent into each measurement chamber.

In some embodiments the device further comprises a second liquid reagent chamber and wherein each of the measurement chambers is fluidically connected to the second liquid reagent chamber, and wherein the device comprises a corresponding number of second liquid reagent chamber conduit valves for independent control of the flow of second liquid reagent into each measurement chamber.

In some embodiments the second liquid reagent chamber conduit is fluidically connected to the first liquid reagent chamber conduit thereby providing one or more combined conduits fluidically connecting both the first liquid reagent chamber and second liquid reagent chamber to each measurement chamber.

In some embodiments the flow of any one or more of the sample liquid, first liquid reagent and/or the second liquid reagent into each of the measurement chambers can be independently controlled to regulate the residence time of each liquid in each of the measurement chambers. In one embodiment the flow of the sample liquid into each of the measurement chambers can be independently controlled to regulate the residence time of the sample liquid in each of the measurement chambers. In one embodiment the flow of the first liquid reagent into each of the measurement chambers can be independently controlled to regulate the residence time of the first liquid reagent in each of the measurement chambers. In one embodiment the flow of the second liquid reagent into each of the measurement chambers can be independently controlled to regulate the residence time of the second liquid reagent in each of the measurement chambers.

In some embodiments the flow of any one or more of the sample liquid, first liquid reagent and/or the second liquid reagent is controlled such that the residence time of each liquid is a predetermined period of time. In one embodiment the flow of the sample liquid is controlled such that the residence time of the sample liquid is a predetermined period of time. In one embodiment the flow of the first liquid reagent is controlled such that the residence time of the first liquid reagent is a predetermined period of time. In one embodiment the flow of the second liquid reagent is controlled such that the residence time of the second liquid reagent is a predetermined period of time.

In some embodiments the device further comprises: a mixing zone located between the sample chamber and the measurement chamber and wherein the mixing zone is fluidically connected to both the sample chamber and the measurement chamber.

In some embodiments the mixing zone comprises a mixing chamber, wherein the mixing chamber is fluidically connected to the sample chamber conduit and to the measurement chamber by a mixing chamber conduit.

In some embodiments the device further comprises: a third liquid reagent chamber; a third liquid reagent chamber conduit which fluidically connects the third liquid reagent chamber to the mixing zone, optionally wherein the third liquid reagent chamber conduit connects to the mixing zone in an alternate flow direction to the sample chamber conduit; and a third liquid reagent chamber conduit valve for opening and closing the third liquid reagent chamber conduit.

In some embodiments the flow of the third liquid reagent into the mixing zone can be independently controlled to regulate the residence time of the third liquid reagent in the mixing zone. In some embodiments the flow of the third liquid reagent is controlled such that the residence time of the third liquid reagent is a predetermined period of time.

In some aspects of the invention one or more of the first liquid reagent chamber, the second liquid reagent chamber and the third liquid reagent chamber may be referred to as auxiliary chambers. In one embodiment the first liquid reagent chamber is referred to as an auxiliary chamber. In one embodiment the second liquid reagent chamber is referred to as an auxiliary chamber. In one embodiment the third liquid reagent chamber is referred to as an auxiliary chamber.

In one aspect a method of performing a diagnostic assay may comprise sequentially moving liquid from a sample chamber to a measurement chamber and moving a first liquid reagent into the measurement chamber from an alternate flow direction, the method including: filling the sample chamber with sample liquid; moving sample liquid from the sample chamber to the measurement chamber; retaining the sample liquid in the measurement chamber for a predetermined period of time, moving a first liquid reagent from a first liquid reagent chamber into the measurement chamber in an alternate flow direction to the sample chamber liquid and taking a measurement, optionally wherein the first liquid reagent is retained in the measurement chamber for a predetermined period of time.

In some methods the first liquid reagent is removed from the measurement chamber before the measurement is taken.

In some embodiments the method further comprises a step of moving liquid from a second liquid reagent chamber to the measurement chamber in an alternate flow direction to sample liquid.

In one aspect a method of performing a diagnostic assay may comprise sequentially moving liquid from a sample chamber to a measurement chamber and moving a first and second liquid reagent into the measurement chamber from an alternate flow direction, the method including: filling the sample chamber with sample liquid; moving sample liquid from the sample chamber to the measurement chamber; retaining the sample liquid in the measurement chamber for a predetermined period of time, moving a first liquid reagent from a first liquid reagent chamber into the measurement chamber in an alternate flow direction to the sample liquid; moving a second liquid reagent from a second liquid reagent chamber into the measurement chamber in an alternate flow direction to the sample liquid and performing a measurement, optionally wherein the first and second liquid reagents are each retained in the measurement chamber for a predetermined period of time.

In some methods the second liquid reagent is removed from the measurement chamber before the measurement is taken.

In one aspect a method of performing a diagnostic assay may comprise sequentially moving liquid from a sample chamber to a measurement chamber and moving a first and second liquid reagent into the measurement chamber from an alternate flow direction, the method including: filling the sample chamber with sample liquid; moving sample liquid from the sample chamber to the measurement chamber; retaining the sample liquid in the measurement chamber for a predetermined period of time, moving a first liquid reagent from a first liquid reagent chamber into the measurement chamber in an alternate flow direction to the sample liquid; moving a second liquid reagent from a second liquid reagent chamber into the measurement chamber in an alternate flow direction to the sample liquid; moving a further volume of the first liquid reagent from the first liquid reagent chamber into the measurement chamber in an alternate flow direction to the sample liquid and performing a measurement, optionally wherein the first and second liquid reagents are each retained in the measurement chamber for a predetermined period of time.

In some methods the flow direction of the first liquid reagent and/or the second liquid reagent is at least ninety degrees to the flow direction of the sample liquid into the measurement chamber, preferably wherein the flow direction of the first liquid reagent and/or second liquid reagent is opposite to the flow direction of the sample liquid.

In some embodiments the methods further comprise a step of mixing the sample liquid with one or more additional reagents before moving the sample liquid into the measurement chamber.

In some methods the sample liquid is mixed in a mixing zone with a third liquid reagent from a third liquid reagent chamber.

### METHODS OF THE INVENTION

The invention also provides a method of implementing any of the methods of the invention on any device of the invention as set out above.

In some embodiments the first liquid reagent is any liquid composition suitable for use as a washing liquid in immunoassays, for example a wash buffer. In some embodiments the first liquid reagent is a liquid comprising one or more reagents selected from the list of a pH buffer (e.g. PBS, Tris, carbonate/bicarbonate, HEPES, MOPS, MES), a salt solution (e.g. NaCl, KCl, MgCl2), a detergent (e.g. Tween 20, Tween 80, Triton-X, CHAPS) and a stabilizer/blocking agent (e.g. BSA, casein).

In some embodiments the first liquid reagent is Tris-buffered saline (TBS) and phosphate-buffered saline (PBS) containing 0.05% (v/v) Tween®-20.

In some embodiments the second liquid reagent is a detection reagent for use in immunoassays. In some embodiments the second liquid reagent comprises one or more reagents selected from DAB (3, 3′-diaminobenzidine), metal-enhanced DAB, AEC (3-amino-9-ethylcarbazole), BCIP (5-bromo-4-chloro-3-indolyl phosphate), NBT (nitro-blue tetrazolium chloride), TMB (3,3′,5,5′-tetramethylbenzidine), ELF (enzyme-labelled fluorescence) and OPD (ophenylenediamine dihydrochloride), preferably wherein the second liquid reagent comprises 3,3′,5,5′-Tetramethylbenzidine (TMB).

In some embodiments the predetermined period of time is from 1 to 180 seconds (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101,102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 or 180 seconds).

In some embodiments the predetermined period of time is from 1 to 60 seconds (e.g. 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 seconds).

In some embodiments the predetermined period of time is from 10 to 30 seconds (e.g. 10, 11, 12, 13, 14, 15, 16, 17, 18, 19, 20, 21, 22, 23, 24, 25, 26, 27, 28, 29 or 30 seconds).

In some embodiments the predetermined period of time is from 60 to 180 seconds (e.g. 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 or 180 seconds).

In one aspect, a cartridge is provided for a microfluidic system, where the reagents are stored, integrated within the cartridge in sealed reservoirs so as not to flow into the microfluidic device until dictated by operation. This allows for long term storage of cartridges containing reagents, while protecting the reagents and microfluidic device from contamination and degradation. An advantage of the devices described herein includes a valve in a microfluidic system having simple construction geometry, allowing cost-effective manufacture of valve features and components. Another advantage is a very small volume, appropriate to the smaller volumes of fluid being employed in microfluidic devices, as compared to any non-integrated off-device valve.

In one aspect, a liquid handling device may comprise a sample chamber for receiving a sample, a measurement chamber for performing one or more measurements on the sample wherein the measurement chamber comprises a reaction zone and a first liquid reagent chamber fluidically connected to the measurement chamber in an alternate flow direction to the sample chamber. a variable pressure source conduit for connecting the measurement chamber to a variable pressure source; a sample chamber conduit which fluidically connects the sample chamber to the measurement chamber; a sample chamber conduit valve for opening and closing the sample chamber conduit; a respective measurement chamber conduit for each measurement chamber, wherein each respective measurement chamber conduit fluidically connects the respective measurement chamber to the measurement chamber; and a respective measurement chamber conduit valve for opening and closing each respective measurement chamber conduit.

The liquid handling device allows a first or second liquid reagent to be transferred to the measurement chamber in an alternate flow direction to the sample liquid. This configuration allows liquid reagents (such as buffers or detection reagents) to be transferred to the measurement chamber through separate conduits which have not previously had sample liquid flowed through them. This configuration allows rapid, precise and controllable quenching of reactions and/or biological interactions in the measurement chamber. The use of conduits with different flow directions also provides reduced contamination of each liquid during different method steps (i.e. reduced contamination of sample liquid in a wash step). This may not be readily achievable with known fluid handling devices, such as conventional microfluidic devices.

The liquid handling device allows a sample to be transferred from the sample chamber into the measurement chamber by reducing the pressure in the measurement chamber relative to the sample chamber. Precise control of the volume of sample transferred into the measurement chamber is possible by controlling the pressure change in the measurement chamber. In the measurement chamber, the sample liquid may react or mix with a reagent. The device allows the sample to be held in the measurement chamber for as long as necessary, for example for a duration of time needed to complete a reaction with a reagent. This may not be readily achievable with known fluid handling devices, such as conventional microfluidic devices.

The sample may be held in the measurement chamber while a measurement is performed, for example as part of a diagnostic test such as an immunoassay. Again, precise control of the volume of sample transferred into the measurement chamber and residence time in the measurement chamber are possible.

The liquid handling device may be provided with or without a variable pressure source. That is to say that a variable pressure source may be integrated into the liquid handling device, but is preferably reversibly connected to the liquid handling device and as such may be provided separately.

A variable pressure source is a pressure source that can apply or provide both positive and negative pressure changes. For example, the variable pressure source may be a syringe and may be controlled by a stepper motor. Other variable pressure sources and ways of controlling variable pressure sources are known to the skilled person.

The liquid handling device is not limited to having only one measurement chamber or only one variable pressure source.

The measurement chamber may be arranged to receive a fluid from the sample chamber when the sample chamber conduit valve is open and a negative pressure change is applied to the one or more measurement chambers.

The reagent chambers may store reagents such as an antibody or antigen binding portion thereof or protein solution, antibody or antigen binding portion thereof or protein powder, buffer solution, an enzyme substrate such as 3,3′,5,5′-tetramethylbenzidine “TMB,” and so on, for mixing or reacting with the sample in order to facilitate a measurement on the sample in the measurement chamber, for example to perform a diagnostic test on the sample.

The reagents in the reagent chambers may be readily mixed with the sample by controlling pressure changes in the liquid handling device. By providing a measurement chamber surrounded by one or more reagent chambers, the device facilitates complex mixing or washing operations, for example operations with multiple steps each requiring precise volume control and timing that may not be readily achieved using known fluid handling devices.

The one or more measurement chambers may comprise a first measurement chamber for performing a first measurement on the sample and a second measurement chamber for performing a second measurement on the sample. As such, the liquid handling device may comprise a first measurement chamber conduit which fluidically connects the first measurement chamber to the sample chamber or the mixing chamber; a second measurement chamber conduit which fluidically connects the second measurement chamber to the sample chamber or the mixing chamber; a first measurement chamber conduit valve for opening and closing the first measurement chamber conduit; and a second measurement chamber conduit valve for opening and closing the second measurement chamber conduit.

As such, a single liquid handling device may be configured to receive only one sample in the sample chamber yet perform multiple measurements or diagnostic tests for determining multiple properties of the sample.

The one or more reagent chambers may comprise one or more first dedicated reagent chambers for reagents to be used only in a diagnostic test to be performed in the first measurement chamber, one or more second dedicated reagent chambers for reagents to be used only in a diagnostic test to be performed in the second measurement chamber, and one or more shared reagent chambers for reagents to be used in the diagnostic tests to be measured in both the first and second measurement chambers.

Ordinarily, separate measurement chambers would each require their own separate reagent sources, however, by providing a shared reagent chamber that provides a reagent, such as a buffer solution, common to two separate diagnostic tests or measurements, a more compact liquid handling device may be provided. The same dedicated reagent chambers store reagents, such as specific antibodies or proteins, that may be selectively mixed with the sample for particular diagnostic tests or measurements, providing the device with a broader range of functionality.

The liquid handling device comprising one or more reagent chambers may further comprise a mixing chamber for mixing the sample with a reagent from one of the one or more reagent chambers. As such, the device also comprises a mixing chamber conduit, wherein the mixing chamber conduit fluidically connects the mixing chamber to the measurement chamber; and a mixing chamber conduit valve for opening and closing the mixing chamber conduit.

Once a reagent is combined with the sample, the resulting combination may be shuttled (transferred back and forth) between the measurement chamber and mixing chamber to accelerate mixing of the reagent and sample (homogenise the reagent and sample) or accelerate dissolution of the reagent in the sample or other liquid.

The liquid handling device may further comprise a waste chamber and a waste chamber conduit, wherein the waste chamber conduit fluidically connects the waste chamber to the measurement chamber and/or the mixing chamber.

The waste chamber may be used to safely store excess sample and/or reagents, for example after the liquid handling device has been used to perform a measurement on the sample. Further, sample may be overprovided to the mixing chamber, and then transferred into another chamber such as a measurement chamber in a precise quantity, while the excess sample is expelled to the waste chamber. The precisely measured sample can then be transferred to a different chamber with a precise known volume.

The liquid handling device may further comprise a waste chamber conduit valve for opening and closing the waste chamber conduit. Alternatively, the waste chamber conduit may fluidically connect the waste chamber to the mixing chamber via the measurement chamber. Thus, sample can be transferred directly from the measurement chamber to the waste chamber after a measurement has been performed.

At least one of the one or more measurement chambers may comprise a plurality of electrodes. The plurality of electrodes may be for performing an electrochemical measurement. Alternatively, or in addition, at least one of the one of more measurement chambers may comprise an element for performing an optical measurement, such as a window.

Each conduit valve may be a pinch valve. A pinch valve may be operated by an external actuator that selectively applies pressure to the pinch valve to open or close it. Optionally, the conduit valves may be configured in a circular array, so that they can be operated by an actuator with a circular array of actuation elements. A pinch valve is a valve which uses a pinching effect to obstruct fluid flow.

The conduit valves of the devices described above may be configured such that only one valve is open at any given time. The conduit valves of the devices described above may be closed by default.

The chambers of the liquid handling device may comprise gas exchange holes for allowing air or any other ambient gas to enter and exit each chamber to balance a pressure change resulting from liquid (such as a sample or reagent) entering the respective chambers, although this is not essential.

The liquid handling device may be made from conventional materials known to the skilled person such as acrylic, glass, silicon, or polydimethylsiloxane (PDMS), using conventional methods such as chemical etching, laser etching, routing or moulding.

Pressure changes are applied via a variable pressure source conduit of the liquid handling device, and may be applied using a variable pressure source, such as a syringe or any other means suitable for applying positive and negative pressure changes, connected to the variable pressure source conduit. The variable pressure source conduit may be connected to the measurement chamber or mixing chamber. Alternatively, the variable pressure source conduit may be connected to another suitable part of the device to allow for precise control of the pressure changes throughout the device.

The method of operating a liquid handling device, wherein the liquid handling device comprises one or more reagent chambers as described above, may further comprise opening the reagent chamber conduit valve corresponding to one of the one or more reagent chambers; reducing a pressure in the mixing chamber relative to the one of the one or more reagent chambers; and closing the reagent chamber conduit valve corresponding to the one of the one or more reagent chambers.

Thus, a reagent may be transferred from a reagent chamber to the mixing chamber.

The method may further comprise, prior to reducing a pressure in the mixing chamber relative to the one of the one or more reagent chambers, increasing a pressure in the mixing chamber relative to the one of the one or more reagent chambers in order to transfer a liquid in the mixing chamber, such as a sample, into the one of the one or more reagent chambers. Thus, if the one of the one or more reagent chambers comprises a dried or powdered reagent, a liquid in the mixing chamber can be used to suspend or dissolve the reagent and then transfer it into the measurement chamber.

When the liquid handling device comprises a mixing chamber as described above, the method of operating a liquid handling device may further comprise opening the mixing chamber conduit valve; increasing a pressure in the measurement chamber relative to the mixing chamber; reducing a pressure in the measurement chamber relative to the mixing chamber and closing the mixing chamber conduit valve.

Thus, a mixture, such as a mixture of a sample and a reagent, may be shuttled between the measurement chamber and mixing chamber or between the one or more reagent chambers and mixing chamber to accelerate mixing of the reagent and sample (e.g. homogenise reagent and sample) or accelerate dissolution of the reagent in the sample.

The method may further comprise repeating increasing a pressure in the mixing chamber and reducing a pressure in the mixing chamber one or more times before closing the mixing chamber conduit valve.

When the liquid handling device comprises a waste chamber, waste chamber conduit and waste chamber conduit valve as described above, the method of operating a liquid handling device may further comprise closing the one of the respective measurement chamber conduit valves; opening the waste chamber conduit valve; increasing a pressure in the measurement chamber relative to the waste chamber; and closing the waste chamber conduit valve. The method may further comprise closing the one of the respective measurement chamber conduit valves.

Thus, liquid in the measurement chamber may be transferred to the waste chamber where it may be safely stored, for example after the liquid handling device has been used to perform a measurement on the sample.

When the liquid handling device comprises a waste chamber and waste chamber conduit fluidically connecting the waste chamber to the mixing chamber via the measurement chamber the method of operating a liquid handling device may further comprise increasing a pressure in the mixing chamber relative to the waste chamber after performing a measurement on the sample.

Thus, liquid in the mixing chamber may be transferred to the waste chamber where it may be safely stored, for example after the liquid handling device has been used to perform a measurement on the sample.

When the one or more measurement chambers comprise a plurality of electrodes, the method of operating a liquid handling device may further comprise performing an electrochemical measurement on a sample using the plurality of electrodes.

When each conduit valve of the liquid handling device is a pinch valve, the method of operating a liquid handling device may further comprise opening or closing at least one of the pinch valves by operating an actuator. The pinch valves may be configured to only open one-at-a-time (i.e. only one pinch valve is open at any one time).

As will be understood, the methods described above can be performed in combination with each other, and in many different orders or multiple times, as required for a given diagnostic test. The order of each method is not limited to the order in which the features are presented above, and one method need not be completed before another method is begun. For example, a method for mixing may be performed after a sample and reagent are introduced into the measurement chamber but before at least a portion of the sample is transferred to the measurement chamber.

In another aspect, a method of performing a diagnostic test using a liquid handling device as described above comprises filling the sample chamber with a sample and performing one or more of the methods described above. Optionally, the liquid handling device comprises one or more reagent chambers and each of the one or more reagent chambers comprises a respective reagent for the diagnostic test.

In another aspect, a method of operating a liquid handling device may comprise opening of the third liquid reagent chamber conduit valves and increasing or reducing the pressure in the mixing chamber relative to the third liquid reagent chamber by a predetermined amount, thereby enabling transfer of a metered volume of a liquid between the mixing chamber and the third liquid reagent chamber. As such, the liquid handling device comprises a mixing chamber; a third liquid reagent chamber; a third liquid reagent chamber conduit, wherein the third liquid reagent chamber conduit fluidically connects the third liquid reagent chamber to the mixing chamber and a third liquid reagent chamber conduit valve for opening and closing the third liquid reagent chamber conduit. The use of predetermined pressure changes enables transfer of precise volumes of liquid.

Increasing or reducing the pressure in the measurement chamber or mixing chamber relative to an auxiliary chamber by a predetermined amount may comprise applying a predetermined pressure change for a predetermined period of time.

When the pressure in the mixing chamber is increased, transfer of a metered volume of a liquid from the mixing chamber an auxiliary chamber is enabled. When the pressure in the mixing chamber is reduced, transfer of a metered volume of a liquid from the auxiliary chamber to the mixing chamber is enabled.

In another aspect, a computer program may comprise computer-executable instructions which, when executed by a system, cause the system to perform the any of the methods described above.

In another aspect, a system may comprise a processor configured to execute a computer program comprising computer-executable instructions which, when executed by a system, cause the system to perform any of the methods described above.

A system may be a point-of-care system or diagnostic system and/or may be for performing a diagnostic test on a sample.

The system may further comprise one or more of a variable pressure source configured to connect to a liquid handling device; a variable pressure source controller to control the variable pressure source; an actuator configured to selectively open or close each of the plurality of pinch valves and a liquid handling device as described above. The processor may be configured to control the variable pressure source controller to control the variable pressure source in accordance with any of the above described methods. The system may further comprise memory for storing the computer program.

In some embodiments, an alternative flow direction may be at least ninety degrees to a first flow direction when measured in the same horizontal plane of the device. In some embodiments, an alternative flow direction may be from 90 to 180 degrees to a first flow direction when measured in the same horizontal plane of the device. In some embodiments, an alternative flow direction may be 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179 or 180 degrees to a first flow direction when measured in the same horizontal plane of the device.

### Methods of Performing Immunoassays

The present invention is applicable to methods of performing immunoassays with a sensor comprising means of magnetically retaining beads at the surface of the sensor. In some embodiments, the present invention may be employed in one or more of the following areas: immunosensors, most notably in the context of point-of-care testing; electrochemical immunoassays; whole blood immunoassays and single-use cartridge based immunoassays. As will be appreciated by the skilled person, the general concept disclosed herein is applicable to many immunoassay methods and platforms.

The methods of the invention are applicable to various biological sample types (e.g., blood, plasma, serum, urine, interstitial fluid and cerebrospinal fluid). The present invention is applicable a variety of immunoassays including both sandwich and competitive immunoassays.

### Sandwich Immunoassays

Immunoassays are often used for the detection of a specific analyte within a sample. For example, pairs of antibodies that can bind to an analyte to form a sandwich that is detectable by means of an enzyme or particulate label on one of the antibodies are well known and available for a wide range of different analytes of interest. For example, antibodies to a particular biomarker, such as testosterone or cortisol, may be used to test levels of these substances in the saliva, blood or urine samples.

The presence of the antibody-analyte sandwich can be detected by various means including by the use of electrochemical measurements. There have also been some reports of assays that utilise an enzyme substrate to amplify the assay signal, and thus improve assay sensitivity, by generating an insoluble precipitate that is detectable by an electrochemical measurement.

Many assays for biomarkers or analytes of interest have been carried out on electrode devices. A typical sandwich assay uses a primary capture antibody or antigen binding portion thereof and a secondary signal antibody or antigen binding portion thereof to bind to different sites of an analyte. Conventional sandwich assays rely on the immobilisation of the capture antibody or antigen binding portion thereof and removal of excess signal antibody or antigen binding portion thereof before readings are taken. Signal generated is proportional to the amount of analyte sandwiched. No target analyte results in signal noise only. Sandwich assays are used for example in pregnancy test devices.

In some embodiments of the invention, the biological sample, e.g., whole blood sample, is collected and then altered by adding a reagent comprising magnetically susceptible beads into the biological sample. The beads preferably include an antibody or antigen binding portion thereof to the analyte of interest immobilized on the outer surface thereof.

The magnetically susceptible bead concentration employed may vary according to the arrangement of the assay. In some embodiments, the sample is altered with the magnetically susceptible beads to provide a dissolved bead concentration of at least 5 μg per μl of sample, for example, at least 10 μg per μl of sample, or at least 15 μg per μl of sample. Once the sample has been mixed with magnetically susceptible beads, preferably include an antibody or antigen binding portion thereof to the analyte of interest immobilized on the outer surface thereof, it is possible to perform an immunoassay, e.g., an electrochemical immunoassay, on the altered sample to determine the concentration of an analyte. In some embodiments of the invention, at least about 10,000 beads are used for each assay. In some embodiments, the dissolved bead volume is less than about 1% of the total sample assay volume and is optionally less than about 0.1% of the total sample assay volume.

In the measurement step, in some embodiments, once the sandwich is formed between the analyte-specific antibody or antigen binding portion thereof immobilized on the bead, the analyte and signal antibodies, a magnetic field is applied to attract the beads in a position to allow them to be washed. The sample is subsequently washed to a waste chamber while leaving the beads (for example substantially all of the beads in the sample) retained in a fixed position within the cartridge (optionally at the electrode, or optionally at a location spatially separated from the electrode), followed by exposing the sandwich on the magnetically susceptible beads to a substrate capable of reacting with an enzyme to form a product capable of electrochemical detection. One exemplary format is an electrochemical enzyme-linked immunosorbent assay.

### Biological Samples

In the present invention, the sample liquid may be any suitable biological sample comprising diagnostic biomarkers of interest. In some embodiments, the biological sample may be a whole blood sample, a serum sample, a plasma sample, a saliva sample, a biopsy sample (such as a healthy tissue sample or a tumour sample), a urine sample, a semen sample, a tear sample, a sputum sample, a sweat sample, a mucous sample, a fecal sample, a gastric fluid sample, an abdominal fluid sample, an amniotic fluid sample, a cyst fluid sample, a peritoneal fluid sample, a spinal fluid sample or a synovial fluid sample, although whole blood samples are particularly preferred. In a preferred embodiment of the invention the biological sample is a whole blood sample. The method may include a step of obtaining or providing the biological sample, or alternatively the sample may have already been obtained from a subject, for example in ex vivo methods.

Biological samples obtained from a subject can be stored until needed. Suitable storage methods include freezing the biological sample immediately upon collection, within 2 hours of collection or up to two weeks after collection. Maintenance at −80° C. can be used for long-term storage. One or more suitable preservatives may be added, or the sample may be collected in a tube containing one or more suitable preservatives. Preferably the sample is analysed immediately (or as soon as possible) following collection.

Methods of the invention may comprise additional steps carried out on biological samples. The sample liquid is considered to be representative of the biomarker status of the biomarkers or analytes of interest in different patient disease states. Hence the methods of the present invention may use quantitative data on biomarkers or analytes of interest, to determine the presence, absence or severity of different disease states. The methods disclosed herein are particularly useful for analysis of human blood.

The sample may be additionally processed prior to determining the status of the biomarkers or analytes of interest. The sample may be enriched (for example to increase the concentration of the biomarkers or analytes of interest being quantified), centrifugation or dilution. In other embodiments, the samples do not undergo any pre-processing and are used unprocessed (such as whole blood).

In some embodiments of the invention, the biological sample may be fractionated or enriched for particular biomarkers prior to detection and quantification (i.e. measurement). The step of fractionation or enrichment can be any suitable pre-processing method step to increase the concentration of a biomarker of interest in the sample. For example, the steps of fractionation and/or enrichment may comprise centrifugation and/or filtration to remove cells or unwanted analytes or fractions from the sample, or to increase the concentration of biomarkers of interest in a particular blood fraction. Such methods are known to the skilled person and may be used to enrich the sample for any biomarkers of interest.

The methods of the invention may be carried out on one test sample from a subject. Alternatively, a plurality of test samples may be taken from a subject, for example at least 2, at least 3, at least 4 or at least 5 samples from a subject. Each sample may be subjected to a single assay to quantify one of the biomarker panel members, or alternatively a sample may be tested for all of the biomarkers being quantified. Each sample may be subjected to a separate analysis using a method of the invention, or alternatively multiple samples from a single subject undergoing diagnosis could be included in the method.

A “sample(s)”, “one or more samples”, sample liquid, biological sample, or “sample(s) of interest” are terms used interchangeably in singular or plural form and are not intended to be limited to any particular quantity and, as used herein, may be any molecule or substance that the user wishes to gather information from. A sample may become larger or smaller (e.g., by way of inflation, dilution, fractionation or partitioning) in size, volume or content during the performance of an assay. Accordingly, a sample may be amplified and/or subdivided one or more times during the performance of an assay. In some embodiments, the sample comprises, or is suspected of comprising, biomarkers or analytes of interest.

A “liquid”, as used herein, is any aqueous or lipophilic phase capable of flowing freely. The liquid may further comprise one or more reagents, reaction components or samples of interest selected from cells (including any eukaryotic or prokaryotic cells, including but not limited to cells selected from humans, animals, plants, fungi, bacteria, viruses, protozoa, yeasts, molds, algae, rickettsia, and prions); proteins, peptides, antibodies, nucleic acid sequences, oligonucleotide probes, polymerase enzymes, buffers, dNTPs, organic and inorganic chemicals, and fluorescent dyes.

The embodiments are not limited to a microfluidic scale but applications on other, for larger scales, are equally envisaged.

### Magnetic Capture

In some embodiments of the invention, the immunosensor comprises an electrode and has a magnet, for example a permanent magnet or an electromagnet, positioned in close proximity to (preferably below) the sensor. The magnetic immunosensor of the invention provides a field of greater than about 0.1 Tesla (for example 0.1 Tesla, 0.2 Tesla, 0.3 Tesla, 0.4 Tesla or 0.5 Tesla, preferably 0.4 Tesla) and is capable of attracting and retaining magnetically susceptible beads from a range of about 0.05 mm to about 5 mm in the region of the electrode.

The magnetic field can be measured, for example, as the magnetic field on a substantially flat surface area of a magnet. The skilled person will recognise that permanent magnets can include ferrite or aluminum nickel cobalt (AlNiCo) magnets, which typically exhibit fields of 0.1 to 1 Tesla. Other high-field permanent magnets comprised of alloys of rare earth elements (e.g., neodymium alloys and samarium cobalt (SmCo) alloys) exhibit fields in excess of 1 Tesla, e.g., greater than 1.2 Tesla or greater than 1.4 Tesla.

In some embodiments the magnetic field can be modulated by physically actuating the permanent or electromagnet between one or more positions located at different physical distances from the desired location (for example the sensor, or a spatially separate location for the purposes of washing). For example, the magnetic field can be modulated by physically actuating the permanent or electromagnet between two positions, a first position which is distant from the desired location (for example the sensor, or a spatially separate location for the purposes of washing) and a second position which is proximate to the desired location (for example the sensor, or a spatially separate location for the purposes of washing), wherein in the second position the magnet provides a sufficiently large magnetic field to capture the magnetically susceptible beads at the desired location (for example the sensor, or a spatially separate location for the purposes of washing).

In another embodiment, the magnet comprises an electromagnet in which the magnetic field is produced by the flow of electric current. The electric current may be provided by a analytical device, into which the cartridge comprising the sensor is inserted and with which the sensor is in electrical contact.

### Magnetically Susceptible Beads

In some embodiments of the invention, the biological sample, e.g., blood sample, is mixed with magnetically susceptible beads. The magnetically susceptible beads may be comprised of any material known in the field that is susceptible to movement by a magnet (for example a permanent magnet or an electromagnet) utilized in or in conjunction with the immunosensor cartridge of the present invention. As such, the terms “magnetic” and “magnetically susceptible” with regard to beads can be used interchangeably.

In some embodiments of the invention, the beads include a magnetic core, which preferably is completely or partially coated with a coating material. The magnetic core may comprise a ferromagnetic, paramagnetic or a superparamagnetic material. In preferred embodiments, the magnetically susceptible beads comprise a ferrite core and an outer polymer coating. In a preferred embodiment, the magnetically susceptible beads are Dynabeads®. Dynabeads® magnetic beads are uniform, non-porous, superparamagnetic, monodispersed and highly crosslinked polystyrene microspheres consisting of an even dispersion of magnetic material throughout the bead. The magnetic material within the Dynabeads® magnetic beads consists of a mixture of maghemite (gamma-Fe2O3) and magnetite (Fe3O4). The iron content (Fe) of the beads is 12% by weight in Dynabeads® magnetic beads M-280 and 20% by weight in Dynabeads® magnetic beads M-450. The Dynabeads® magnetic beads are coated with a thin polystyrene shell which encases the magnetic material, and prevents any leakage from the beads or trapping of ligands in the bead interior. The shell also protects the target from exposure to iron while providing a defined surface area for the adsorption or coupling of various molecules.

The magnetic core may comprise one or more of Fe, Co, Mn, Ni, metals comprising one or more of these elements, ordered alloys of these elements, crystals comprised of these elements, magnetic oxide structures, such as ferrites, and combinations thereof. In other embodiments, the magnetic core may be comprised of magnetite, maghemite, or divalent metal-ferrites wherein the metal is, for example, Cu, Fe, Ni, Co, Mn, Mg, or Zn or combinations of these materials.

Suitable materials for the coating include synthetic and biological polymers, copolymers and polymer blends, and inorganic materials. Polymer materials may include various combinations of polymers of acrylates, siloxanes, styrenes, acetates, alkylene glycols, alkylenes, alkylene oxides, parylenes, lactic acid, and glycolic acid. Biopolymer materials include starch or similar carbohydrate. Inorganic coating materials may include any combination of a metal, a metal alloy, and a ceramic. Examples of ceramic materials may include hydroxyapatite, silicon carbide, carboxylate, sulfonate, phosphate, ferrite, phosphonate, and oxides of Group IV elements of the Periodic Table of Elements.

In other embodiments of the invention, the magnetic beads may be formed from a non-magnetic substrate, for example, of a material selected from the group consisting of polystyrene, polyacrylic acid and dextran, upon which a suitable magnetic coating is placed.

Any appropriately-sized magnetically susceptible bead capable of being positioned with the magnet of the present invention may be utilized, taking into account the dispersability requirements for the magnetically susceptible beads. In preferred embodiments, at least 50% (measured by % weight) of the magnetically susceptible beads in the assay are retained at the electrode surface (or spatially distant washing area surface). In some embodiments, at least 60%, 70%, 80%, 90% or 95% (measured by % weight) of the magnetically susceptible beads in a given sample are retained at the electrode surface (or spatially distant washing area surface). As used herein, the term “spatially distant” or “spatially distant position” refers to any suitable area where magnetically susceptible beads may be retained (for example for the purpose of washing) that does not substantially overlap with the electrode or detection area.

In some exemplary embodiments, the average particle size of the magnetically susceptible beads may range from about 0.01 μm to about 10 μm, about 0.05 μm to about 10 μm, about 0.1 μm to about 10 μm, about 0.2 μm to about 10 μm, about 0.5 μm to about 10 μm, about 0.8 μm to about 10 μm, about 1 μm to about 10 μm, about 2 μm to about 10 μm, about 5 μm to about 10 μm, about 0.01 μm to about 5 μm, about 0.05 μm to about 5 μm, about 0.1 μm to about 5 μm, about 0.2 μm to about 5 μm, about 0.5 μm to about 5 μm, about 0.8 μm to about 5 μm, about 1 μm to about 5 μm, about 2 μm to about 5 μm, about 0.01 μm to about 2 μm, about 0.05 μm to about 2 μm, about 0.1 μm to about 2 μm, about 0.2 μm to about 2 μm, about 0.5 μm to about 2 μm, about 0.8 μm to about 2 μm, about 1 μm to about 2 μm, about 0.05 μm to about 1 μm, about 0.1 μm to about 1 μm, about 0.2 μm to about 1 μm, about 0.5 μm to about 1 μm, about 0.8 μm to about 1 μm, about 0.05 μm to about 0.8 μm, about 0.1 μm to about 0.8 μm, about 0.2 μm to about 0.8 μm, about 0.5 μm to about 0.8 μm, about 0.05 μm to about 0.5 μm, about 0.1 μm to about 0.5 μm, about 0.2 μm to about 0.5 μm, about 0.05 μm to about 0.2 μm, about 0.1 μm to about 0.2 μm, about 0.05 μm to about 0.1 μm. Preferably, the magnetically susceptible beads have an average particle size of about 1 μm.

As used herein, the term “average particle size” refers to the average longest dimension of the particles, e.g., beads, for example the diameter for spherical particles, as determined by methods well-known in the field. The particle size distribution of the magnetically susceptible beads is preferably unimodal, although beads with polymodal distributions may also be used. While use of a spherical magnetically susceptible bead is preferred, in other embodiments, other bead shapes and structures, e.g., ovals, sub-spherical, cylindrical and other irregular shaped particles, are within the meaning of the term “beads” and “microparticles” as used herein.

Commercial sources for magnetically susceptible bead preparations include Invitrogen (Carlsbad, California, U.S.A.) by Life Technologies, Ademtech (Pessac, France), Chemicell GmbH (Berlin, Germany), Bangs Laboratories, Inc.® (Fishers, IN) and Seradyn, Inc. (Indianapolis, IN). Many of the commercially available products incorporate surface functionalization that can be employed to immobilize antibodies (for example, IgG) on the bead surfaces. Functionalisation of the beads can include carboxyl, amino or streptavidin-modified magnetically susceptible beads.

The magnetically susceptible beads are preferably coated with an antibody or antigen binding portion thereof to an analyte that is a cardiovascular marker, e.g., cardiac troponin I, troponin T, a troponin complex, proBNP, NT-proBNP, human chorionic gonadotropin, BNP, creatine kinase, creatine kinase subunit M, creatine kinase subunit B, creatine kinase MB (CK-MB), myoglobin, myosin light chain or modified fragments thereof, among others. In a preferred embodiment, the cardiovascular marker is troponin I or troponin T. In another preferred embodiment, the cardiovascular marker is proBNP, NT-proBNP.

In addition, markers for other indications can be utilized. Further exemplary analytes include, but are not limited to, beta-HCG, TSH, ultra hTSH II, TT3, TT4, FT3, FT4, myeloperoxidase, D-dimer, CRP, NGAL, PSA, LH, FSH, galectin-3, prolactin, progesterone, estradiol, DHEA-S, AFP, CA 125 II, CA 125, CA 15-3, CA 19-9, CA 19-9XR, CEA, thyroxine (T4), triiodothyronine (T3), T-uptake, Tg, anti-Tg, anti-TPO, ferritin, Cortisol, insulin, HBsAg, HCV Ag/Ab combo, HCV core Ag, anti-HCV, AUSAB (anti-HBs), CORE, CORE-M, SHBG, iPTH, theophylline, sirolimus, tacrolimus, anti-HAV, anti-HAV IgM, HAVAB, HAVAB-M, HAVAB-M2.0, HAVAB-G, HAVAB 2.0, HAVAB 2.0 Quant, IgM, CMV IgM, CMV IgG, a-2-microglobulin, digitoxin, HBe, anti-HBe, HBeAg, HIV I/2gO, HIV Ag/Ab combo, testosterone, SCC, vitamin B 12, folate, syphilis, anti-HBc, aibella IgG, aibella IgM, homocysteine, MPO, cytomegaloviais (CMV) IgG Avidity, toxo IgG avidity, toxo IgG, toxo IgM, C-peptide, vitamin D, HTLV I/II, total ahCG, progesterone, estradrogen, prolactin, myoglobin, tPSA, fPSA, carbamazepine (CBZ), digoxin, gentamicin, NAPA, phenytoin, phenobarbital, valproic acid, vancomycin, procaine, quinidine, tobramycin, methamphetamine (METH), amphetamine (AMPH), barbituates, benzodiazepine, Cannabis, cocaine, methadone, opiates, PCP, acetaminophen, ethanol, salicylates, tricyclics, holoTc, anti-CCP, HbAlc, barbs-U, among others. In certain embodiments of the invention, the antibody or antigen binding portion thereof is to a low-abundance analyte in the sample. Abbreviated names above will be familiar to the skilled person.

The magnetic immunosensor and methods of the present invention preferably also comprise a second antibody or antigen binding portion thereof, which is an enzyme-linked antibody or antigen binding portion thereof, also referred to herein as a signal antibody or antigen binding portion thereof. In some embodiments, the enzyme-linked antibody or antigen binding portion thereof is in the form of a liquid reagent, which also may comprise the magnetically susceptible beads that are employed in the present invention, as discussed below. Both the bead-immobilized and enzyme-linked antibodies can be monoclonal, polyclonal, fragments thereof and combinations thereof. In addition, one or more of the antibodies can be labeled with various labels including a radiolabel, enzyme, chromophore, fluorophore, chemiluminescent species, ionophore, electroactive species and others known in the immunoassay art. Where the second antibody or antigen binding portion thereof is labeled with an enzyme, it is preferably ALP, horseradish peroxidase (HRP), or glucose oxidase. In other embodiments, the analyte is labeled with fluorescein, ferrocene, p-aminophenol, or derivatives thereof. In a preferred embodiment, the second antibody or antigen binding portion thereof is enzyme-linked to horseradish peroxidase (HRP).

In some embodiments, the magnetically susceptible beads are homogeneously mixed with the sample. In still other embodiments, the magnetically susceptible beads may be less homogeneously mixed with the sample; however, one object of the invention is to optimise the position and concentration of the beads relative to the electrode. The skilled person will recognise that the magnetically susceptible beads of the present invention may be added to the biological sample prior to introduction into the magnetic immunosensor device, such as, for example, as an integral part of a blood collection device or as a standard manual addition step. However, for the convenience of the user and to assure a quality assay, the magnetically susceptible beads are preferably included within the immunosensor cartridge, for example in one or more of the liquid storage capsules described above.

In some embodiments of the invention, the sample, e.g., whole blood sample, is collected and then modified by combining with a reagent comprising the magnetically susceptible beads. In addition to the magnetically susceptible beads, the reagent may further include one or more of: beads for reducing leukocyte interference, a leukocidal reagent, buffer, salt, surfactant, stabilizing agent, simple carbohydrate, complex carbohydrate and various combinations thereof. The reagent can also include an enzyme-labeled antibody or antigen binding portion thereof (e.g., the above-described labeled antibody or antigen binding portion thereof) to the analyte. In some embodiments of the invention, the additional components required for the assay are contained in combination in a single reagent. In some embodiments of the invention, at least two additional components required for the assay are contained in combination in a single reagent and other components required for the assay are contained in separate reagents. In other embodiments of the invention, the additional components required for the assay are contained individually in separate reagents.

In some embodiments, the magnetically susceptible beads are used to modify the biological sample, e.g., blood, in a first container or location, and then the sample is passed to a second container or location that includes the capture and signal antibodies. In some embodiments, the magnetically susceptible beads are contained in solution and mixed with the biological sample, and the resulting modified sample is introduced into the magnetic immunosensor cartridge. For example, a blood sample may be mixed with the magnetically susceptible beads to form a modified sample, which is then introduced into the device. In certain embodiments, the magnetic immunosensor device, e.g., cartridge, includes a capsule that contains a liquid comprising the magnetically susceptible beads, which may be mixed with a biological sample in the device and then processed substantially as described herein to form an assay (e.g., sandwich assay) for analyte detection.

In addition, any immunoassay format known in the art may be modified to include the magnetically susceptible beads of the present invention, for example, by adding the beads in a sample pre-treatment step. The pre-treatment may be accomplished, for example, by incorporating the beads in a blood collection device, in a separate vessel, or may take place in the immunoassay device itself by incorporation of the beads as part of the assay method.

In some embodiments of the invention, the beads are mobile and thereby capable of interacting with an analyte. After binding to the analyte of interest, magnetic forces are used to concentrate the beads for the purposes of washing (either at the electrode or at a spatially separate location from the electrode) and then at the electrode for the purpose of measurement. One advantage of using mobile beads according to the present invention is that their motion in the sample or fluid accelerates binding reactions, making the capture step of the assay faster.

In some embodiments of the invention, additives may be included in the magnetic immunosensor device or used in conjunction with the assay. In some embodiments, an anticoagulant can be added. For example, heparin may be added to improve performance in cases where the sample was not collected in a heparinized tube or was not properly mixed in a heparinized tube. Any suitable amount of heparin may be added so that fresh unheparinized blood will remain uncoagulated during the assay cycle of the cartridge, typically in the range of 2 to 20 minutes. In still other embodiments, one or more of proclin, DEAE-dextran, tris buffer, and lactitol can be added as reagent stabilizers. In further embodiments, a surfactant such as polysorbate 20, also known as Tween® 20, can be added to reduce binding of proteins to plastic, which is a preferred material for the cartridge housing of the magnetic immunosensing device. The addition of a surfactant also facilitates the even coating of reagents on plastic surfaces and minimizes the crystallization of sugars (e.g., lactitol). In other embodiments of the invention, an antibacterial agent or biocide (e.g., sodium azide) may be added to inhibit bacterial growth.

### Computer Methods

The above-described methods can be performed in combination with each other, and in many different orders or multiple times, as required for a given diagnostic test. One method need not be completed before another method is performed.

The described methods may be implemented by a diagnostic system using computer executable instructions. A computer program product or computer readable medium may comprise or store the computer executable instructions. The computer program product or computer readable medium may comprise a hard disk drive, a flash memory, a read-only memory (ROM), a CD, a DVD, a cache, a random-access memory (RAM) and/or any other storage media in which information is stored for any duration (e.g. for extended time periods, permanently, brief instances, for temporarily buffering, and/or for caching of the information). A computer program may comprise the computer executable instructions. The computer readable medium may be a tangible or non-transitory computer readable medium. The term “computer readable” encompasses “machine readable”.

Thus, also disclosed is a computer program comprising computer-executable instructions which, when executed by a diagnostic system, cause the diagnostic system to perform any of the methods described above.

### EXAMPLES

### Example 1—Magnet Actuation Used to Spread Beads on the Electrode to Increase Signal Response

Human pooled plasma (Lit-heparin) supplemented with 625 ng/ml of analyte (recombinant NT-pro-BNP) is measured using a standard procedure. Magnetic particles (1 um epoxy beads) coated with capture antibody or antigen binding portion thereof were mixed with plasma samples and sample diluent (25 mM aqueous TBS) containing bovine serum albumin (5%), calcium chloride (20 mM), magnesium chloride (20 mM), Proclin 300 (0.05%), and Tween 20 (0.1%) and detection antibody labelled with horseradish peroxidase (HRP).

The reaction mixture was incubated in tube for 2 minutes at room temperature after which the magnetic particles were removed by magnetic field and washed with assay wash buffer containing a standard concentration of tween-20 in PBS (10 mM). The washed magnetic particles were driven in the cartridge at a specific flow rate (2 μl/sec) and localised on an electrode.

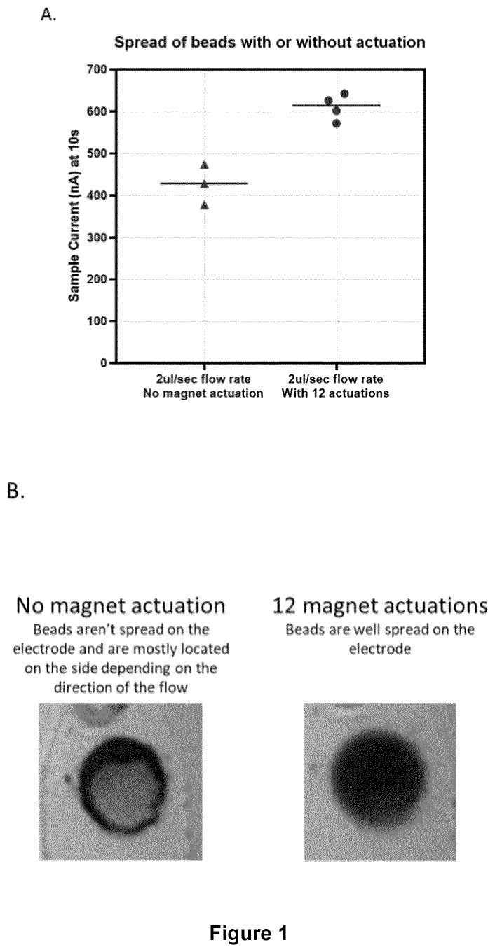

A magnetic field (0.4 Tesla) was applied ON and OFF 12 times by using magnet actuation in comparison to magnetic field applied without actuation of the magnet. A 3,3′,5,5′-Tetramethylbenzidine (TMB) solution was introduced in the electrode chamber. The assay response recorded for each sample (current after 60 seconds chronoamperometry at −50 mV with IR compensation) is plotted to compare signal obtain with or without actuation (FIG. 1). As shown in FIG. 1, the assay method provides a 1.5× signal increase using the optimised method.

### Example 2—Wash with and without Magnet Actuation—Optimisation of a Bead Assay Wash Using Magnet Actuation

Human plasma (EDTA) containing zero analyte was measured in parallel using a standard procedure in flow cell format wherein only the magnet actuation process is varied.

Identical wash volumes (470 μl) were tested under standardised assay conditions. Plasma samples were mixed with sample diluent containing detection antibody labelled with horseradish peroxidase (HRP) in a 1:1 ratio and magnetic particles coated with capture antibody. The reaction mixture was incubated in a fluidic channel for 5 minutes at 30° C. after which the magnetic particles were removed by magnetic field and washed with assay wash buffer (varying only the use or no use of magnet actuation process during the wash).

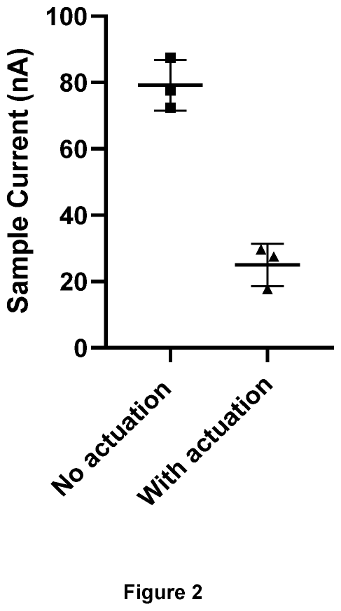

The washed magnetic particles were resuspended and transported to a clean flow cell where the beads are pulled down on a 2.5 mm electrode and a detection solution introduced. The assay response current recorded using chronoamperometry at −50 mV for zero and 100 ng/L analyte samples using each washing process (varying magnet actuation) showed that using a controlled mechanical magnet actuation during the wash process reduce the unspecific binding of the assay defined by the background measure when containing zero analyte (FIG. 2). FIG. 2 shows the background signal is reduced by a factor of 4 by using the improved wash method.

### Example 3—Wash Volume Reduction by Magnet Actuation Process—Optimisation of a Bead Assay Wash Using Magnet Actuation

Human plasma (EDTA) containing zero analyte and the same plasma supplemented with 100 ng/L analyte were measured in parallel using a standard procedure in flow cell format wherein only the wash volume is varied.

Identical actuation method is used under standardised assay conditions. Plasma samples were mixed with sample diluent containing detection antibody labelled with horseradish peroxidase (HRP) in a 1:1 ratio and magnetic particles coated with capture antibody. The reaction mixture was incubated in a fluidic channel for 5 minutes at 30° C. after which the magnetic particles were removed by magnetic field and washed with assay wash buffer using sequential mechanical magnet actuations (varying only the total wash volume used 470 μl, 260 μl, 160 μl). The washed magnetic particles were resuspended and transported to a clean flow cell where the beads are pulled down on a 2.5 mm electrode and a detection solution introduced.

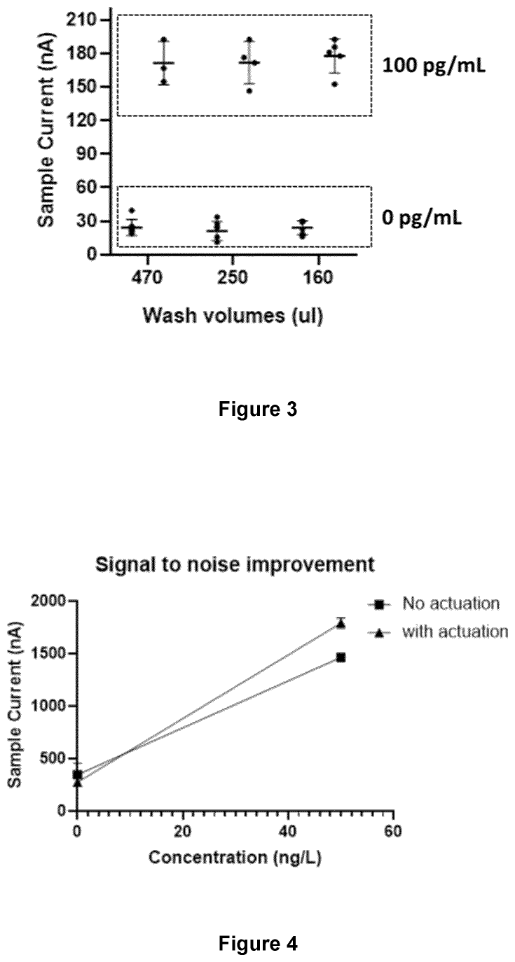

The assay response current recorded using chronoamperometry at −50 mV for zero and 100 ng/L analyte samples using the same washing process (varying the total wash Volume) showed that using a controlled mechanical magnet actuation during the wash process allow not only having lower background response, but more so reducing the volumes necessary to wash the assay from 470 μl to 160 μl (FIG. 3). FIG. 3 shows the wash volume is reduced by a factor of 3 by using the improved wash method. This allows for smaller volumes of wash liquid to be stored on board of the cartridge and therefore reduces the cartridge footprint needed. Smaller cartridges are more suitable for point-of-care testing devices, thereby meaning this method is an improved method for use with such devices.

### Example 4—Signal Improvement Using Magnet Actuation—Optimisation of Signal to Noise Using Magnet Actuation

Human plasma (EDTA) containing zero analyte was used in parallel to the same sample supplemented with 50 ng/L of analyte and measured in parallel using a standard procedure in flow cell format wherein only the magnet actuation process is varied prior measurement.

Identical reagents were tested under standardised assay conditions. Plasma samples were mixed with sample diluent containing detection antibody labelled with horseradish peroxidase (HRP) in a 1:1 ratio and magnetic particles coated with capture antibody. The reaction mixture was incubated in a fluidic channel for 5 minutes at 30° C. after which the magnetic particles were removed by magnetic field and washed with assay wash buffer.

The washed magnetic particles were resuspended and transported to a clean flow cell where the beads are pulled down on a 2.5 mm electrode and a detection solution introduced (we compare using or no use of magnet actuation) prior reading. The assay response current recorded using chronoamperometry at −50 mV for zero and 50 ng/L analyte samples using or not using magnet actuation showed that using a controlled mechanical magnet actuation increase the specific signa and reduce the unspecific interactions which is reflected in increased performance of the assay (FIG. 4).

### Example 5—Beads Resuspension Using Air-Liquid Interfaces—Optimisation of Bead Resuspension Using Air Liquid Interfaces

Human plasma (EDTA) containing zero analyte was supplemented with 100 ng/L of analyte and measured in parallel using a standard procedure in flow cell format wherein only the number of air-liquid interfaces used is varied.

Identical reagents were tested under standardised assay conditions. Plasma samples were mixed with sample diluent containing detection antibody labelled with horseradish peroxidase (HRP) in a 1:1 ratio and magnetic particles coated with capture antibody. The reaction mixture was incubated in a fluidic channel for 5 minutes at 30° C. after which the magnetic particles were removed by magnetic field and washed with assay wash buffer. The washed magnetic particles were resuspended and transported to a clean flow cell varying the number of air-liquid interfaces used for transferring the beads.

In the case where one interface was used during resuspension and transfer of the magnetic beads, the flow cell was emptied with air flow and a single volume of wash buffer was used to resuspend and transfer the magnetic beads to the sensor. In the case where two interfaces were used during resuspension and transfer of the magnetic beads, the flow cell was emptied with air flow and a first and a second volumes of wash buffer were used to resuspend and transfer the magnetic beads to the sensor. Air flow was used to generate the air-liquid interfaces between the first and second volumes of wash buffer.

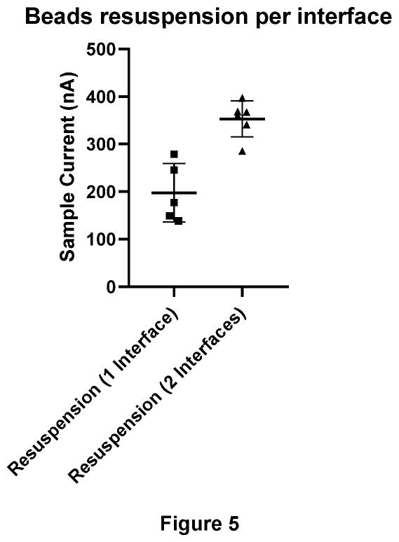

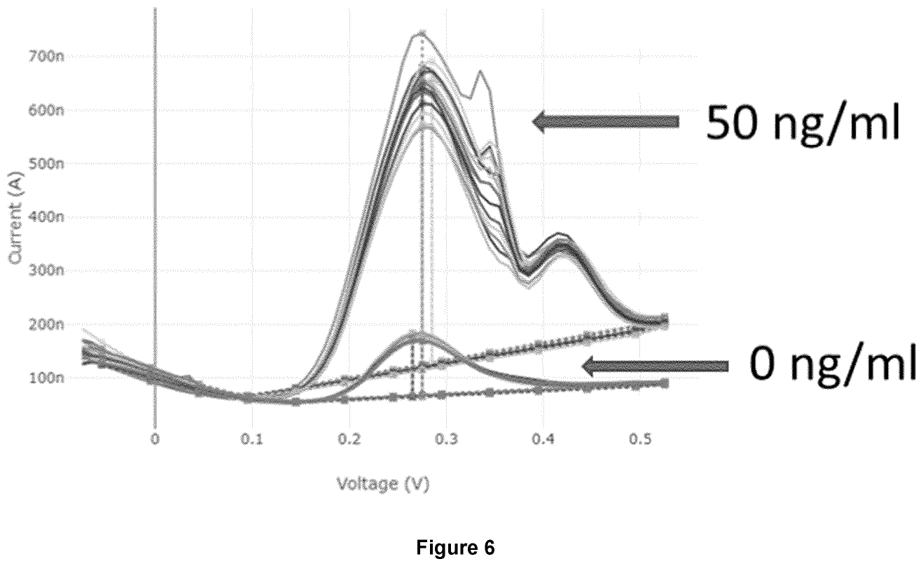

The beads are pulled down on a 2.5 mm electrode and a detection solution introduced. The assay response current recorded using chronoamperometry at −50 mV for zero and 100 ng/L analyte samples using each interfacial iteration (varying interfaces) showed that increasing the number if interfaces increases the signal of the assay which is reflected in the increase of number/spread of beads on the electrode surface (FIG. 5).

### Example 6—Establishing Ability to Measure Precipitating TMB on Magnetic Beads Using DPV

Troponin free serum was used as a matrix for the experiment and standard experimental procedures were followed.

Magnetic particles coated with capture antibody were mixed with troponin free serum and sample diluent (25 mM aqueous TBS) containing bovine serum albumin (5%), sodium chloride (400 mM), and detection antibody labelled with horseradish peroxidase (HRP). The reaction mixture was incubated in a well plate for 5 minutes at 25° C. after which the magnetic particles were removed by magnetic field and washed with assay wash buffer PBS-T 0.1%.

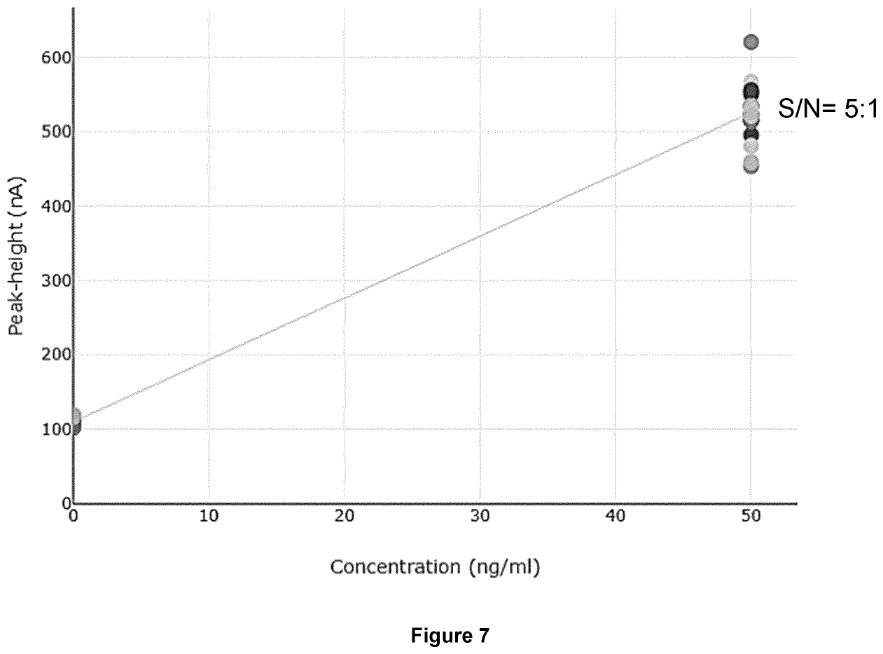

The washed magnetic particles were resuspended on a well and precipitating 3′,3′,5,5′-Tetramethylbenzidine (TMB) solution introduced and incubated for 1 min. Afterwards the TMB was removed by pulling the magnetic particles on the bottom of the well plate using the magnet. Then the beads were resuspended and loaded on a separate well and pulled down on the electrode surface. The assay response recorded for two concentrations 0 and 50 ng/ml was recorded using differential pulse voltammetry (DPV) peak height signal and the signal-to-noise ratio (S/N) (FIG. 7) is displayed. These results confirm that the TMB substrate has precipitated on the magnetic beads.

According to the proposed method having the TMB precipitating on the magnetic beads allows for the detection reaction to be stopped by using additional wash and optionally resuspension steps. The additional wash step improves the signal to noise ratio by reducing the background without significantly affecting the detection signal.

### Example 7—Multiplex Assays

Magnetic beads can be coated with antibodies for two or more different target analytes (for example NT-proBNP and troponin). Sample will be mixed (or premixed) with a diluent and then incubated with a mixture of different second (i.e. detection) antibodies against each of the target analytes. For example, for a first analyte (e.g. NT-proBNP) the detection antibody may be labelled with alkaline phosphatase and for a second analyte (e.g. troponin) the detection antibody may be labelled with HRP. This solution will then be mixed with the magnetic particles and incubated for a period of time (as described herein) and at a specific temperature (as described herein). The beads will then be captured on the surface of the electrode with the use of a magnet.

The beads with the analyte and the labelled secondary antibodies will be washed by resuspension and pulled down. After washing, two substrates will be added in sequence to the beads: 1) One precipitating substrate for the first enzyme (e.g. an alkaline phosphatase substrate such as BCIP/NBT) will be added and incubated for a period of time. This will react and locally deposit precipitate in areas of the bead where the first analyte (e.g. NT-proBNP) has been captured. Then the beads will be washed so the excess substrate will be removed and only the precipitate formed on the beads will remain. Subsequently a suitable substrate for the second enzyme (e.g. an HRP substrate such as precipitating TMB) will be added and, like the first enzyme substrate, it will react and precipitate only in areas where the second analyte (e.g. troponin) has been captured. The beads will undergo one final wash and then will be transferred to the detection electrode.

Then an electrochemical detection technique will be used (preferably differential pulse voltammetry, DPV) and two characteristic peaks will be obtained for each of the precipitating reagents as these have a distinct electrochemical profile. The signal generated is proportional to the amount of each analyte. In a similar fashion, beads can be coated with multiple precipitating electrochemical mediators that have a unique electrochemical profile to create more multiplexed options for parallel detection of different analytes.

The present invention is further described by the following numbered embodiments:
1) A method for measuring an analyte of interest in a biological sample comprising:
   combining said biological sample with a composition comprising:
      magnetically susceptible beads conjugated to a first antibody or antigen binding portion thereof capable of binding said analyte of interest; and
      a second antibody or antigen binding portion thereof capable of binding said analyte of interest conjugated to an enzyme;
   retaining the magnetically susceptible beads on an electrode using a magnetic field;
   contacting the magnetically susceptible beads with a substrate for the enzyme, wherein the enzyme substrate is converted by the enzyme into an electroactive molecule; and
   obtaining an electrochemical measurement using said electrode.
2) A method for measuring one or more analytes of interest in a biological sample comprising:
   combining said biological sample with a composition comprising:
      magnetically susceptible beads conjugated to one or more antibodies or antigen binding portions thereof, wherein each antibody or antigen binding portion thereof is capable of binding one of said analytes of interest; and
      one or more second antibodies or antigen binding portions thereof conjugated to an enzyme, wherein each second antibody or antigen binding portion thereof is capable of binding one of said analytes of interest;
   retaining the magnetically susceptible beads on an electrode using a magnetic field;
   contacting the magnetically susceptible beads with one or more substrates for the enzymes, wherein the enzyme substrates are converted by the enzymes into electroactive molecules; and
   obtaining an electrochemical measurement using said electrode.
3) The method according to embodiment 2, wherein the method is capable of detecting at least 2, 3, 4, 5, 6, 7, 8, 9 or 10 different analytes of interest.
4) The method according to embodiment 2 or embodiment 3, wherein each magnetically susceptible bead is conjugated to multiple different antibodies or antigen binding portions thereof, wherein each antibody or antigen binding portion thereof is capable of binding one of said analytes of interest.
5) The method according to embodiment 2 or embodiment 3, wherein the magnetically susceptible beads comprise different sets of magnetically susceptible beads, wherein each set of magnetically susceptible beads is conjugated to a different antibody or antigen binding portion thereof, wherein each antibody or antigen binding portion thereof is capable of binding one of said analytes of interest.
6) The method for measuring an analyte of interest in a biological sample according to any preceding embodiment, wherein the enzyme substrate is converted by the enzyme into a soluble electroactive molecule at the electrode.
7) The method for measuring an analyte of interest in a biological sample according to any one of embodiments 1 to 5, wherein the enzyme substrate is converted by the enzyme into an electroactive molecule precipitated on the magnetically susceptible beads.
8) The method for measuring an analyte of interest in a biological sample according to any preceding embodiment, further comprising:
   incubating the combined biological sample and composition such that the one or more first and/or one or more second antibodies bind the analyte of interest.
9) The method for measuring an analyte of interest in a biological sample according to any preceding embodiment, further comprising:
   retaining the magnetically susceptible beads in a fixed position using a magnetic field; and
   washing the magnetically susceptible beads.
10) The method of embodiment 9, wherein the fixed position is located at the electrode.
11) The method of embodiment 9, wherein the fixed position is a position spatially distant from said electrode, optionally wherein the spatially distant position is a blank or non-functional electrode.
12) The method for measuring an analyte of interest in a biological sample according to any one of embodiments 1 to 9, further comprising:
   retaining the magnetically susceptible beads in a first position using a magnetic field; and
   washing the magnetically susceptible beads by modulating the magnetic field such that the magnetically susceptible beads are retained in a second position.
13) The method for measuring an analyte of interest in a biological sample according to any preceding embodiment, wherein the magnetic field is generated by a fixed magnet.
14) The method for measuring an analyte of interest in a biological sample according to any preceding embodiment, wherein the magnetic field is generated by an electromagnet.
15) The method for measuring an analyte of interest in a biological sample according to any one of embodiments 12 to 14, wherein the magnetic field is modulated by physically actuating the magnet.
16) The method for measuring an analyte of interest in a biological sample according to any one of embodiments 12 to 14, wherein the magnetic field is modulated by controlling the current in an electromagnet.
17) The method for measuring an analyte of interest in a biological sample according to any one of embodiments 9 to 15, wherein the magnetic field is located within a microfluidic device having a flow conduit and wherein the magnetic field is modulated by actuating the magnet in a direction perpendicular to the direction of flow within said flow conduit.
18) The method for measuring an analyte of interest in a biological sample according to any one of embodiments 9 to 14 or 16, wherein the magnetic field is located within a microfluidic device having a flow conduit and wherein the magnetic field is modulated by controlling the current in an electromagnet.
19) The method for measuring an analyte of interest in a biological sample according to any one of embodiments 12 to 18 wherein the magnetically susceptible beads are moved between the first position and the second position at least 20 times, for example at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times or at least 19 times.
20) The method for measuring an analyte of interest in a biological sample according to any one of embodiments 12 to 18 wherein the magnetically susceptible beads are moved between the first position and the second position twice, 3 times, 4 times, 5 times, 6 times, 7 times, 8 times, 9 times, 10 times, 11 times, 12 times, 13 times, 14 times, 15 times, 16 times, 17 times, 18 times, 19 times or 20 times, preferably 12 times.
21) The method for measuring an analyte of interest in a biological sample according to any one of embodiments 12 to 20, wherein the magnetic field is modulated such that the magnetically susceptible beads are moved periodically between the first position and the second position.
22) The method for measuring an analyte of interest in a biological sample according to embodiment 21, wherein the period between the magnetically susceptible beads being in the first position and in the second position is between about 0.01 seconds to about 5 seconds, about 0.05 seconds to about 5 seconds, about 0.1 seconds to about 5 seconds, about 0.2 seconds to about 5 seconds, about 0.3 seconds to about 5 seconds, about 0.4 seconds to about 5 seconds, about 0.5 seconds to about 5 seconds, about 1 seconds to about 5 seconds, about 2 seconds to about 5 seconds, about 3 seconds to about 5 seconds, about 4 seconds to about 5 seconds, about 0.01 seconds to about 4 seconds, about 0.05 seconds to about 4 seconds, about 0.1 seconds to about 4 seconds, about 0.2 seconds to about 4 seconds, about 0.3 seconds to about 4 seconds, about 0.4 seconds to about 4 seconds, about 0.5 seconds to about 4 seconds, about 1 seconds to about 4 seconds, about 2 seconds to about 4 seconds, about 3 seconds to about 4 seconds, about 0.01 seconds to about 3 seconds, about 0.05 seconds to about 3 seconds, about 0.1 seconds to about 3 seconds, about 0.2 seconds to about 3 seconds, about 0.3 seconds to about 3 seconds, about 0.4 seconds to about 3 seconds, about 0.5 seconds to about 3 seconds, about 1 seconds to about 3 seconds, about 2 seconds to about 3 seconds, about 0.01 seconds to about 2 seconds, about 0.05 seconds to about 2 seconds, about 0.1 seconds to about 2 seconds, about 0.2 seconds to about 2 seconds, about 0.3 seconds to about 2 seconds, about 0.4 seconds to about 2 seconds, about 0.5 seconds to about 2 seconds, about 1 seconds to about 2 seconds, about 0.01 seconds to about 1 seconds, about 0.05 seconds to about 1 seconds, about 0.1 seconds to about 1 seconds, about 0.2 seconds to about 1 seconds, about 0.3 seconds to about 1 seconds, about 0.4 seconds to about 1 seconds, about 0.5 seconds to about 1 seconds, about 0.01 seconds to about 0.5 seconds, about 0.05 seconds to about 0.5 seconds, about 0.1 seconds to about 0.5 seconds, about 0.2 seconds to about 0.5 seconds, about 0.3 seconds to about 0.5 seconds, about 0.4 seconds to about 0.5 seconds, about 0.01 seconds to about 0.4 seconds, about 0.05 seconds to about 0.4 seconds, about 0.1 seconds to about 0.4 seconds, about 0.2 seconds to about 0.4 seconds, about 0.3 seconds to about 0.4 seconds, about 0.01 seconds to about 0.3 seconds, about 0.05 seconds to about 0.3 seconds, about 0.1 seconds to about 0.3 seconds, about 0.2 seconds to about 0.3 seconds, about 0.01 seconds to about 0.2 seconds, about 0.05 seconds to about 0.2 seconds, about 0.1 seconds to about 0.2 seconds, about 0.01 seconds to about 0.1 seconds, about 0.05 seconds to about 0.1 seconds, about 0.01 seconds to about 0.05 seconds, preferably about 3 seconds to about 5 seconds, more preferably about 4 seconds.
23) The method for measuring an analyte of interest in a biological sample according to any one of embodiments 9 to 22, wherein the magnetically susceptible beads are washed with a washing solution and/or air.
24) The method for measuring an analyte of interest in a biological sample according to embodiment 23, wherein the magnetically susceptible beads are washed sequentially and separately using both a washing solution and air.
25) The method for measuring an analyte of interest in a biological sample according to any one of embodiments 9 to 24, wherein the magnetically susceptible beads are alternately washed at least twice, at least three times, at least four times or at least five times with a washing solution and air, preferably wherein the magnetically susceptible beads are alternately washed twice with a washing solution and air.
26) The method for measuring an analyte of interest in a biological sample according to any preceding embodiment wherein the biological sample is diluted before combining with the magnetically susceptible beads.
27) The method for measuring an analyte of interest in a biological sample according to any preceding embodiment wherein the electrode is a carbon ink electrode.
28) The method for measuring an analyte of interest in a biological sample according to any preceding embodiment wherein the analyte of interest is brain natriuretic peptide or N-terminal pro-BNP.
29) The method for measuring an analyte of interest in a biological sample according to any one of embodiments 1 to 28 wherein the analyte of interest is cardiac troponin or cardiac troponin subunit I (cTnI).
30) The method for measuring an analyte of interest in a biological sample according to any one of embodiments 2 to 27, wherein the one or more analytes of interest are selected from the list consisting of: brain natriuretic peptide, N-terminal pro-BNP, cardiac troponin and cardiac troponin subunit I (cTnI).
31) The method for measuring an analyte of interest in a biological sample according to any one of embodiments 2 to 27 or 30, wherein the one or more analytes of interest are N-terminal pro-BNP and cardiac troponin subunit I (cTnI).
32) The method for measuring an analyte of interest in a biological sample according to any preceding embodiment wherein the enzyme is horseradish peroxidase (HRP).
33) The method for measuring an analyte of interest in a biological sample according to any one of embodiments 1 to 31 wherein the enzyme is alkaline phosphatase (ALP).
34) The method for measuring an analyte of interest in a biological sample according to any one of embodiments 2 to 33 wherein at least one of the one or more second antibodies or antigen binding portions thereof is conjugated to horseradish peroxidase (HRP) and at least one of the one or more second antibodies or antigen binding portions thereof is conjugated to alkaline phosphatase (ALP).
35) The method for measuring an analyte of interest in a biological sample according to any preceding embodiment wherein the substrate for the enzyme is selected from the list consisting of: 3,3′,5,5′-Tetramethylbenzidine (TMB), 2,2′-Azinobis [3-ethylbenzothiazoline-6-sulfonic acid]-diammonium salt (ABTS), o-phenylenediamine dihydrochloride (OPD), para-nitrophenyl phosphate (PNPP) and BCIP/NBT (a combination of BCIP (5-Bromo-4-chloro-3-indolyl phosphate) and NBT (nitro blue tetrazolium)).
36) The method for measuring an analyte of interest in a biological sample according to embodiment 35, wherein the product of the enzymatic reaction is precipitated, optionally wherein the product is precipitated onto the magnetically susceptible beads.
37) The method for measuring an analyte of interest in a biological sample according to embodiment 35, wherein the substrate is 3,3′,5,5′-Tetramethylbenzidine (TMB) and the product of the enzymatic reaction is precipitated, optionally wherein the product is precipitated onto the magnetically susceptible beads.
38) The method for measuring an analyte of interest in a biological sample according to embodiment 35, wherein the substrate is BCIP/NBT (a combination of BCIP (5-Bromo-4-chloro-3-indolyl phosphate) and NBT (nitro blue tetrazolium)) and the product of the enzymatic reaction is precipitated, optionally wherein the product is precipitated onto the magnetically susceptible beads.
39) The method for measuring an analyte of interest in a biological sample according to any preceding embodiment wherein the electrochemical measurement is indicative of the concentration or amount of the analyte of interest.
40) The method for measuring an analyte of interest in a biological sample according to embodiment 39, wherein the concentration or amount of the analyte of interest is determined by comparison to a reference solution.
41) The method for measuring an analyte of interest in a biological sample according to any preceding embodiment wherein the electrochemical measurement is an amperometric, voltametric, potentiometric, impedimetric, or electrochemical impedance spectroscopic measurement, preferably a chronoamperometric measurement.
42) The method for measuring an analyte of interest in a biological sample according to any preceding embodiment wherein the electrochemical measurement is differential pulse voltammetry (DPV).
43) The method according to any preceding embodiment, wherein the method of measuring is a sandwich immunoassay.
44) The method according to any preceding embodiment, wherein the at least 50% (for example, at least 60%, at least 70%, at least 80%, at least 90% or at least 95% of the beads) measured by % weight are retained at the surface of the electrode.
45) A method for measuring an analyte of interest in a biological sample comprising:
   combining said biological sample with a composition comprising:
      magnetically susceptible beads conjugated to a first antibody or antigen binding portion thereof capable of binding said analyte of interest; and
      a second antibody or antigen binding portion thereof capable of binding said analyte of interest conjugated to an enzyme;
   retaining the magnetically susceptible beads on an electrode using a magnetic field;
   contacting the magnetically susceptible beads with a substrate for the enzyme, wherein the enzyme substrate is converted by the enzyme into a precipitated electroactive molecule; and
   obtaining an electrochemical measurement using said electrode.
46) A method for measuring an analyte of interest in a biological sample comprising:
   combining said biological sample with a composition comprising:
      magnetically susceptible beads conjugated to one or more antibodies or antigen binding portions thereof, wherein each antibody or antigen binding portion thereof is capable of binding one of said analytes of interest; and
      one or more second antibodies or antigen binding portions thereof conjugated to an enzyme, wherein each second antibody or antigen binding portion thereof is capable of binding one of said analytes of interest;
   retaining the magnetically susceptible beads on an electrode using a magnetic field;
   contacting the magnetically susceptible beads with one or more substrates for the enzymes, wherein the enzyme substrates are converted by the enzymes into precipitated electroactive molecules; and
   obtaining an electrochemical measurement using said electrode.
47) The method according to embodiment 46, wherein the method is capable of detecting at least 2, 3, 4, 5, 6, 7, 8, 9 or 10 different analytes of interest.
48) The method according to any one of embodiments 45 or 46, wherein the electroactive molecule is precipitated onto the magnetically susceptible beads.
49) The method according to any one of embodiments 45 to 47, wherein the one or more analytes of interest are N-terminal pro-BNP and cardiac troponin subunit I (cTnI).
50) The method for measuring an analyte of interest in a biological sample according to any one of embodiments 45 to 48 wherein the enzyme is horseradish peroxidase (HRP).
51) The method for measuring an analyte of interest in a biological sample according to any one of embodiments 45 to 48 wherein the enzyme is alkaline phosphatase (ALP).
52) The method for measuring an analyte of interest in a biological sample according to any one of embodiments 46 to 49 wherein at least one of the one or more second antibodies or antigen binding portions thereof is conjugated to horseradish peroxidase (HRP) and at least one of the one or more second antibodies or antigen binding portions thereof is conjugated to alkaline phosphatase (ALP).
53) The method for measuring an analyte of interest in a biological sample according to any one of embodiments 45 to 51 wherein the substrate for the enzyme is selected from the list consisting of: 3,3′,5,5′-Tetramethylbenzidine (TMB), 2,2′-Azinobis [3-ethylbenzothiazoline-6-sulfonic acid]-diammonium salt (ABTS), o-phenylenediamine dihydrochloride (OPD), para-nitrophenyl phosphate (PNPP) and BCIP/NBT (a combination of BCIP (5-Bromo-4-chloro-3-indolyl phosphate) and NBT (nitro blue tetrazolium)).
54) The method according to any preceding embodiment, wherein the magnetically susceptible beads and the biological sample are incubated at a temperature of between 10° C. and 50° C., optionally between 15° C. and 45° C., further optionally between 20° C. and 40° C., further optionally between 20° C. and 30° C., further optionally between 25° C. and 35° C., further optionally at 25° C., further optionally at 30° C., further optionally at 40° C.
55) A composition comprising magnetically susceptible beads conjugated to a first antibody or antigen binding portion thereof capable of binding to an analyte of interest; and
   a second antibody or antigen binding portion thereof capable of binding said analyte of interest conjugated to an enzyme.
56) A composition comprising magnetically susceptible beads conjugated to one or more antibodies or antigen binding portions thereof, wherein each antibody or antigen binding portion thereof is capable of binding an analyte of interest; and
   one or more second antibodies or antigen binding portions thereof conjugated to an enzyme, wherein each second antibody or antigen binding portion thereof is capable of binding one of said analytes of interest.
57) The composition according to embodiment 56 wherein each magnetically susceptible bead is conjugated to multiple different antibodies or antigen binding portions thereof, wherein each antibody or antigen binding portion thereof is capable of binding one of said analytes of interest.
58) The composition according to embodiment 56, wherein the magnetically susceptible beads comprise different sets of magnetically susceptible beads, wherein each set of magnetically susceptible beads is conjugated to a different antibody or antigen binding portion thereof, wherein each antibody or antigen binding portion thereof is capable of binding one of said analytes of interest.
59) The composition according to any one of embodiments 55 to 58, wherein the composition is capable of detecting at least 2, 3, 4, 5, 6, 7, 8, 9 or 10 different analytes of interest.
60) The composition according to any one of embodiments 55 to 59 wherein the analyte of interest is brain natriuretic peptide or N-terminal pro-BNP.
61) The composition according to any one of embodiments 55 to 59 wherein the analyte of interest is cardiac troponin or cardiac troponin subunit I (cTnI).
62) The composition according to any one of embodiments 55 to 61, wherein the one or more analytes of interest are N-terminal pro-BNP and cardiac troponin subunit I (cTnI).
63) The composition according to any one of embodiments 55 to 62 wherein the enzyme is horseradish peroxidase (HRP) or alkaline phosphatase (ALP).
64) The composition according to any one of embodiments 55 to 63 wherein at least one of the one or more second antibodies or antigen binding portions thereof is conjugated to horseradish peroxidase (HRP) and at least one of the one or more second antibodies or antigen binding portions thereof is conjugated to alkaline phosphatase (ALP).
65) The composition according to any one of embodiments 55 to 64 wherein the substrate for the enzyme is selected from the list consisting of: 3,3′,5,5′-Tetramethylbenzidine (TMB), 2,2′-Azinobis [3-ethylbenzothiazoline-6-sulfonic acid]-diammonium salt (ABTS), o-phenylenediamine dihydrochloride (OPD), para-nitrophenyl phosphate (PNPP) and BCIP/NBT (a combination of BCIP (5-Bromo-4-chloro-3-indolyl phosphate) and NBT (nitro blue tetrazolium)).
66) A kit for performing a method according to any one of embodiments 1 to 54, comprising:
   magnetically susceptible beads;
   an immunoassay apparatus comprising an electrode; and
   a magnet positioned proximate to the chip for retaining the magnetically susceptible beads proximate to the electrode.
67) The kit according to embodiment 66, further comprising a means of retaining the magnetically susceptible beads at a separate location spatially distant from the electrode.
68) The kit according to embodiment 67, wherein the means of retaining the magnetically susceptible beads at a separate location spatially distant from the electrode is the same magnet used to retain the magnetically susceptible beads proximate to the electrode.
69) The kit according to embodiment 67, wherein the means of retaining the magnetically susceptible beads at a separate location spatially distant from the electrode is a second magnet configured to retain the magnetically susceptible beads at a separate location spatially distant from the electrode.
70) The kit according to any one of embodiments 66 to 69, wherein the magnet is a permanent magnet or an electromagnet.
71) A method for measuring an analyte of interest in a biological sample comprising:
   combining said biological sample with a composition comprising:
      magnetically susceptible beads conjugated to a first antibody or antigen binding portion thereof capable of binding said analyte of interest; and
      a second antibody or antigen binding portion thereof capable of binding said analyte of interest conjugated to an enzyme;
   retaining the magnetically susceptible beads on an electrode using a magnetic field;
   contacting the magnetically susceptible beads with a substrate for the enzyme, wherein the enzyme substrate is converted by the enzyme into a precipitated electroactive molecule; and
   obtaining an electrochemical measurement using said electrode, wherein the biological sample and the composition are combined at a position spatially distant from said electrode, optionally wherein the spatially distant position is a blank or non-functional electrode.
72) A method for measuring an analyte of interest in a biological sample comprising:
   combining said biological sample with a composition comprising:
      magnetically susceptible beads conjugated to one or more antibodies or antigen binding portions thereof, wherein each antibody or antigen binding portion thereof is capable of binding one of said analytes of interest; and
      one or more second antibodies or antigen binding portions thereof conjugated to an enzyme, wherein each second antibody or antigen binding portion thereof is capable of binding one of said analytes of interest;
   retaining the magnetically susceptible beads on an electrode using a magnetic field;
   contacting the magnetically susceptible beads with one or more substrates for the enzymes, wherein the enzyme substrates are converted by the enzymes into precipitated electroactive molecules; and
   obtaining an electrochemical measurement using said electrode, wherein the biological sample and the composition are combined at a position spatially distant from said electrode, optionally wherein the spatially distant position is a blank or non-functional electrode.
73) The method for measuring an analyte of interest in a biological sample according to any one of embodiments 71 or 72, wherein the magnetically susceptible beads are contacted with one or more substrates for the enzymes at a position spatially distant from said electrode.
74) The method for measuring an analyte of interest in a biological sample according to any one of embodiments 71 to 73, wherein the magnetically susceptible beads are washed at a position spatially distant from said electrode with a washing solution and/or air.
75) The method for measuring an analyte of interest in a biological sample according to any one of embodiments 71 to 74, wherein the magnetically susceptible beads are washed at a position spatially distant from said electrode sequentially and separately using both a washing solution and air.
76) The method for measuring an analyte of interest in a biological sample according to any one of embodiments 71 to 75, wherein the magnetically susceptible beads are alternately washed at a position spatially distant from said electrode at least twice, at least three times, at least four times or at least five times with a washing solution and air, preferably wherein the magnetically susceptible beads are alternately washed at a position spatially distant from said electrode twice with a washing solution and air.
77) The method for measuring an analyte of interest in a biological sample according to any one of embodiments 71 to 76, wherein the biological sample and the composition are combined at a position spatially distant from said electrode and then the magnetically susceptible beads are moved to said electrode to obtain an electrochemical measurement.
78) The method for measuring an analyte of interest in a biological sample according to any one of embodiments 71 to 77, wherein the magnetically susceptible beads are contacted with one or more substrates for the enzymes at a position spatially distant from said electrode and then the magnetically susceptible beads are moved to said electrode to obtain an electrochemical measurement.
79) The method for measuring an analyte of interest in a biological sample according to any one of embodiments 71 to 78, wherein the magnetically susceptible beads are contacted with one or more substrates for the enzymes at a position spatially distant from said electrode and then the magnetically susceptible beads are moved to said electrode to obtain an electrochemical measurement.
80) The method for measuring an analyte of interest in a biological sample according to any one of embodiments 71 to 79, wherein:
   (i) the biological sample and the composition are combined at a position spatially distant from said electrode; and/or
   (ii) the magnetically susceptible beads are retained and washed at a position spatially distant from said electrode with a washing solution and/or air
   and then the magnetically susceptible beads are moved to said electrode to obtain an electrochemical measurement, optionally wherein the spatially distant position for the combining of the biological sample and the composition and the spatially distant position for the washing are the same.
81) The method for measuring an analyte of interest in a biological sample according to any one of embodiments 71 to 80, wherein:
   (i) the biological sample and the composition are combined at a position spatially distant from said electrode,
   (ii) the magnetically susceptible beads are contacted with one or more substrates for the enzymes at a position spatially distant from said electrode; and/or
   (iii) the magnetically susceptible beads are retained and washed at a position spatially distant from said electrode with a washing solution and/or air
   and then the magnetically susceptible beads are moved to said electrode to obtain an electrochemical measurement, optionally wherein the spatially distant position for the combining of the biological sample and the composition, the spatially distant position for the washing and/or the spatially distant position for contacting the magnetically susceptible beads with one or more substrates for the enzymes are the same.

1) A method for measuring an analyte of interest in a biological sample comprising:
combining said biological sample with a composition comprising:
magnetically susceptible beads conjugated to a first antibody or antigen binding portion thereof capable of binding said analyte of interest; and
a second antibody or antigen binding portion thereof capable of binding said analyte of interest conjugated to an enzyme;
retaining the magnetically susceptible beads on an electrode using a magnetic field;
contacting the magnetically susceptible beads with a substrate for the enzyme, wherein the enzyme substrate is converted by the enzyme into an electroactive molecule; and
obtaining an electrochemical measurement using said electrode.

2) The method for measuring an analyte of interest in a biological sample according to claim 1, wherein the enzyme substrate is converted by the enzyme into a soluble electroactive molecule at the electrode.

3) The method for measuring an analyte of interest in a biological sample according to claim 1, wherein the enzyme substrate is converted by the enzyme into an electroactive molecule precipitated on the magnetically susceptible beads.

4) The method for measuring an analyte of interest in a biological sample according to any preceding claim, further comprising:
incubating the combined biological sample and composition such that the first and/or second antibodies bind the analyte of interest.

5) The method for measuring an analyte of interest in a biological sample according to any preceding claim, further comprising:
retaining the magnetically susceptible beads in a fixed position using a magnetic field; and
washing the magnetically susceptible beads.

6) The method of claim 5, wherein the fixed position is:
a. located at the electrode; or
b. a position spatially distant from said electrode, optionally wherein the spatially distant position is a blank or non-functional electrode.

7) The method for measuring an analyte of interest in a biological sample according to any one of claims 1 to 4, further comprising:
retaining the magnetically susceptible beads in a first position using a magnetic field; and
washing the magnetically susceptible beads by modulating the magnetic field such that the magnetically susceptible beads are retained in a second position.

8) The method for measuring an analyte of interest in a biological sample according to any preceding claim, wherein the magnetic field is generated by:
a. a fixed magnet; or
b. an electromagnet.

9) The method for measuring an analyte of interest in a biological sample according to any one of claim 7 or 8, wherein the magnetic field is modulated by physically actuating the magnet.

10) The method for measuring an analyte of interest in a biological sample according to any preceding claim, wherein the magnetic field is located within a microfluidic device having a flow conduit and wherein the magnetic field is modulated by actuating the magnet in a direction perpendicular to the direction of flow within said flow conduit.

11) The method for measuring an analyte of interest in a biological sample according to any one of claims 7 to 10 wherein the magnetically susceptible beads are moved between the first position and the second position at least 20 times, for example at least 5 times, at least 6 times, at least 7 times, at least 8 times, at least 9 times, at least 10 times, at least 11 times, at least 12 times, at least 13 times, at least 14 times, at least 15 times, at least 16 times, at least 17 times, at least 18 times or at least 19 times.

12) The method for measuring an analyte of interest in a biological sample according to any one of claims 5 to 11, wherein the magnetically susceptible beads are washed with a washing solution and/or air.

13) The method for measuring an analyte of interest in a biological sample according to claim 12, wherein the magnetically susceptible beads are washed sequentially and separately using both a washing solution and air.

14) The method for measuring an analyte of interest in a biological sample according to any one of claims 5 to 13, wherein the magnetically susceptible beads are alternately washed at least twice, at least three times, at least four times or at least five times with a washing solution and air, preferably wherein the magnetically susceptible beads are alternately washed twice with a washing solution and air.

15) The method for measuring an analyte of interest in a biological sample according to any preceding claim wherein the electrode is a carbon ink electrode.

16) The method for measuring an analyte of interest in a biological sample according to any preceding claim wherein the analyte of interest is brain natriuretic peptide or N-terminal pro-BNP.

17) The method for measuring an analyte of interest in a biological sample according to any one of claims 1 to 15 wherein the analyte of interest is cardiac troponin or cardiac troponin subunit I (cTnI).

18) The method for measuring an analyte of interest in a biological sample according to any preceding claim wherein the enzyme is horseradish peroxidase (HRP) or alkaline phosphatase (ALP).

19) The method for measuring an analyte of interest in a biological sample according to any preceding claim wherein the substrate for the enzyme is selected from the list consisting of: 3,3′,5,5′-Tetramethylbenzidine (TMB), 2,2′-Azinobis [3-ethylbenzothiazoline-6-sulfonic acid]-diammonium salt (ABTS), o-phenylenediamine dihydrochloride (OPD), para-nitrophenyl phosphate (PNPP) and BCIP/NBT (a combination of BCIP (5-Bromo-4-chloro-3-indolyl phosphate) and NBT (nitro blue tetrazolium)).

20) The method for measuring an analyte of interest in a biological sample according to any preceding claim wherein the electrochemical measurement is an amperometric, voltametric, potentiometric, impedimetric, or electrochemical impedance spectroscopic measurement, preferably a chronoamperometric measurement.

21) The method according to any preceding claim, wherein the method of measuring is a sandwich immunoassay.

22) The method according to any preceding claim, wherein the at least 50% (for example, at least 60%, at least 70%, at least 80%, at least 90% or at least 95% of the beads) measured by % weight are retained at the surface of the electrode.

23) A kit for performing a method according to any one of claims 1 to 22, comprising:
magnetically susceptible beads;
an immunoassay apparatus comprising an electrode; and
a magnet positioned proximate to the chip for retaining the magnetically susceptible beads proximate to the electrode.

24) The kit of claim 23, further comprising a means of retaining the magnetically susceptible beads at a separate location spatially distant from the electrode.

25) The kit of any one of claims 23 to 24, wherein the magnet is a permanent magnet or an electromagnet.
